(19) United States
(12) Patent Application Publication
(10) Pub. No.: US 2004/0229874 A1
(43) Pub. Date: Nov. 18, 2004

(54) 5HT7 Antagonists and inverse agonists

(75) Inventors: Gene Michael Bright, Groton, CT (US); Karen J. Coffman, Pawcatuck, CT (US)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 10/766,741

(22) Filed: Jan. 27, 2004

Related U.S. Application Data
(60) Provisional application No. 60/444,362, filed on Jan. 31, 2003.

(51) Int. Cl. C 07D 4 3/02; A61K 31/497; A61K 31/501; A61K 31/53

(52) U.S. Cl. 514/242; 514/252.01; 514/252.11; 514/252.14; 514/253.01; 514/255.05; 544/182; 544/238; 544/295; 544/357; 544/360

(57) ABSTRACT

The present invention relates to compounds of formula I,

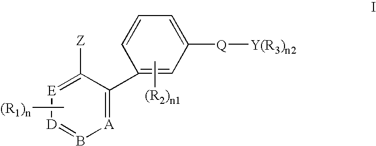

and the pharmaceutically acceptable salts thereof.

These compounds are useful as psychotherapeutic agents.

This application claims the benefit of U.S. Provisional Patent Application Ser. No. 60/444,362, filed Jan. 31, 2003.

The present invention relates to novel serotonin (5HT₇) antagonists and inverse agonists, pharmaceutical compositions containing same, and their medicinal use.

### BACKGROUND OF THE INVENTION

Serotonin 7 receptors are present in the suprachiasmatic nucleus (SCN), the brain region that contains the biological clocks, and their activation leads to a resetting of the clocks as a function of dose and timing of treatment. Such a mechanistic link is evident in numerous paradigms: in in vitro electrophysiological studies of SCN neuronal activity, and in light induced changes in wheel running behavior and nighttime melatonin suppression, in each case activation of 5HT₇ receptors having the potential to modulate both clock function and the clock resetting ability of light. Full antagonists and inverse agonists of the 5HT₇ receptor therefore offer a wide range of chemically useful therapeutics.

Pharmacological effects associated with serotonin receptors include, but are not limited to appetite suppression, thermoregulation, cardiovascular/hypotensive effects, sleep, psychosis, anxiety, depression, nausea, emesis, Alzheimer's disease, Parkinson's disease and Huntington's disease. See, Glennon's article “Serotonin receptors: Clinical Implications”, Neuroscience and Behavioral Reviews, 14, 3547 (1990). Serotonin also plays a role in both the positive and negative symptoms of schizophrenia.

The present invention relates to novel compounds useful for the treatment of diseases or conditions caused by disorders of the serotonin system.

### SUMMARY OF THE INVENTION

The present invention relates to compounds of the formula I

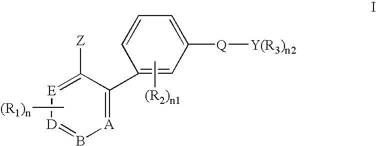

or pharmaceutically acceptable salts thereof wherein

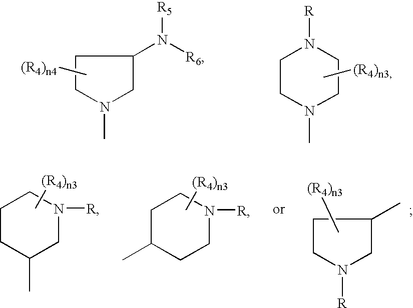

A, B, D, E, are independently CH or N, with at most two of A, B, D and E being N;

each R, R₁, R₂, R₃ and R₄ is independently hydrogen, halo or lower alkyl, which may be unsubstituted or substituted with one to four substituents independently selected from the group consisting of halo, lower alkyl, hydroxy, lower alkoxy, cycloalkyl, cycloalkyl lower alkyl, cycloalkoxy, or cycloalkyl lower alkoxy;

Y is nitrogen containing heteroaryl having 5 to 14 ring atoms and containing at least one ring nitrogen atom and may additionally contain an additional ring heteratom selected from the group consisting of oxygen, nitrogen and sulfur; said heteroaryl containing 5 to 13 ring carbon atoms and up to a total of 20 carbon atoms;

R₅ and R₆ are independently methyl or ethyl;

n is 0 to 4;

n₁ is 0-4;

n₂ is 0-5;

n₃ is 04; and

n₄ is 0-3.

The present invention is also directed to pharmaceutical compositions comprising a therapeutically effective amount of a compound of formula I and a pharmaceutically acceptable carrier.

In another embodiment, the present invention is directed to a method of treating diseases or conditions caused by disorders of the serotonin system which comprises administering to a mammal, for example a human, in need of such treatment a therapeutically effective amount of a compound of formula I.

In still another embodiment, the present invention is directed to the treatment of a disorder or condition selected from the group consisting of depression, anxiety, avoidant personality disorder, premature ejaculation, eating disorder, migraine, premenstrual syndrome, premenstrual dysphonic disorder, seasonal affective disorder, bipolar disorder, jet lag, sleep disorder such as circadian sleep rhythms disorder, sleep deprivation, REM sleep disorders, hypersomnia, parasomnia, sleep-wake cycle disorders, sleep disorder associated with blindness, sleep disorder associated with obesity, narcolepsy, and sleep disorder associated with shift work or irregular work schedules, nocturnal enuresis and restlessleg syndrome in a mammal for example a human, comprising administering to said mammal in need of such treatment a therapeutically effective amount of a compound of formula I.

### DETAILED DESCRIPTION OF THE INVENTION

As used herein, the term “lower alkyl”, when used alone or in combination with other groups, refers to an alkyl group containing one to six carbon atoms. The alkyl group may be straight-chained or branched. Examples include methyl, ethyl, propyl, i-propyl, n-butyl, t-butyl, sec-butyl, i-butyl, pentyl, isopentyl, neopentyl, hexyl, and the like. The preferred alkyl group contains 1 to 3 carbon atoms. The most preferred alkyl group is methyl.

The term “lower alkenyl”, when used alone or in combination with other groups refers to an alkenyl group containing 2 to 6 carbon atoms. It may contain one carbon-carbon double bond or it may contain. 2 or 3 carbon-carbon double bonds. It may be straight chained or branched. Examples include ethenyl, 1-propenyl, 1-butenyl, 2-butenyl, 1-pentenyl, 2-pentenyl, 2-methyl-2-butenyl, and the like.

When used alone or in combination with other groups, the term “lower alkynyl” refers to an alkynyl group containing 2 to 6 carbon atoms. It may be branched or straight-chained. Examples include ethynyl, 1-proynyl, 1-butynyl, 2-butynyl and the like.

As used herein, the term “halo” refers to halogen, such as fluoro, bromo, chloro and iodo.

The term “aryl” refers to an aromatic ring containing only ring carbon atoms. The aryl group contains 6 to 14 ring carbon atoms and up to a total of 20 carbon atoms. The aryl group may be monocyclic, bicyclic or tricyclic, and if bicyclic or tricyclic, the rings are fused. The aryl group may be unsubstituted or substituted with alkyl groups. Examples include phenyl, α-naphthyl, β-naphthyl, anthracenyl, and the like.

“Aralkyl”, as used herein, refers to an aryl group that is connected to the main chain by a bridging alkylene group. Examples include benzyl, phenethyl, phenpropyl, napthylethyl, and the like.

“Heteroaryl”, as used herein, refers to aromatic groups containing one or more heteroatoms (O, S, or N), preferably from one to four heteroatoms. A multicyclic group containing one or more heteroatoms wherein at least one ring of the group is aromatic is a “heteroaryl” group. The heteroaryl groups of this invention can also include ring systems substituted with one or more oxo moieties. Examples of heteroaryl groups are pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, indolyl, benzimidazolyl, benzofuranyl, cinnolinyl, indazolyl, indolizinyl, phthalazinyl, triazinyl, isoindolyl, purinyl, oxadiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl.

The heteroaryl group as used herein, may be monocyclic, bicyclic or tricyclic; if however, it is bicyclic or tricyclic the rings are fused. They also include benzoheterocyclic, especially benzoheterocyclics containing only nitrogen ring atoms. Other examples of heteroaryls include pyrrolyl; pyrazolyl; triazolyl, especially 1, 2, 3 triazolyl or 1, 2, 4-triazolyl; pyrazolyl; isoindolyl; indolyl; indazolyl; carbazole; carbolinyl; thiazolyl; isothiazolyl; oxadiazolyl, e.g., 1, 2, 3-oxadiazolyl, 1,2,4-oxadiazolyl, and 1, 2, 5 oxadiazolyl; 3,4-oxadiazoly; oxatriazolyl, e.g., 1, 2, 3, 4-oxatriazolyl; 1, 2, 3, 4-oxatriazolyl and 1, 2, 3, 5-oxatriazolyl. The most preferred heteroaryl group is benzoimidazolyl, imidazolyl, indolyl, pyrrolyl, triazolyl, pyrazolyl and the like.

Cycloalkyl, as used herein, refers to a cycloalkyl group containing only carbon ring atoms and from 3 to 14 ring carbon atoms. It may be monocyclic, bicyclic, or tricyclic. If the cycloalkyl group contains more than one ring, the rings are fused. Examples include cyclopropyl, cyclobutyl, cyclopentyl, cyclohexyl, cyclooctyl, cycloheotyl, decalinyl, norbronyl, and the like.

The variables n, n₁, n₂, n₃ and n₄ define the number of substituents that may be on the various rings. When n, n₁, n₂, n₃ and n₄ are zero, then the rings are unsubstituted. When n, n₁, n₂, n₃ or n₄ is 1, the ring is mono-substituted. If they are two then the rings are disubstituted. If the rings contain more than one substituent, they may be the same or different.

The preferred values of Z are

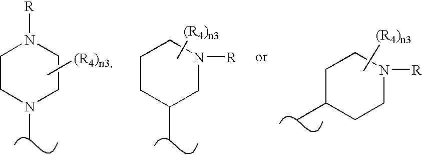

It is even more preferred that Z is

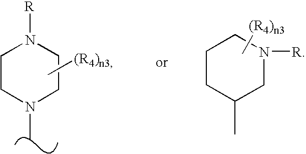

It is to be understood, that with respect to Z, the lines attached to the ring that are connected to just a ring atom refers to the position of the Z ring which is attached to

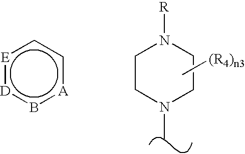

For example, when Z is then the structure of formula I becomes

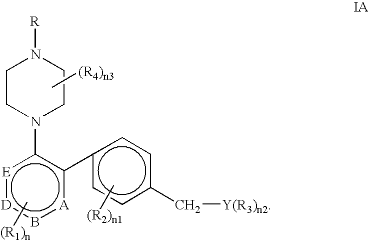

It is preferred that n₃ and n₄ are zero or 1, and most preferably that n₃ and n₄ are zero. If R₄ is other than hydrogen, it is preferred that it is lower alkyl, especially unsubstituted alkyl and most preferably methyl. In the most preferred embodiment n₃ and n₄ are independently zero.

The preferred values of R are hydrogen or methyl. When Z is a piperazinyl, it is most preferred that R is lower alkyl, especially methyl. When Z is a piperidinyl, it is preferred that R is lower alkyl, especially methyl. On the other hand, when Z is a piperidine, it is preferred that R is lower alkyl, e.g. methyl, or especially hydrogen.

It is preferred that at most one of E, D, B or A is nitrogen and it is most preferred that all of them are CH. It is preferred that n is 0 or 1, that is, the

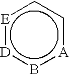

ring is unsubstituted or monosubstituted. If monosubstituted, it is preferred that R₁ is alkyl and more preferably unsubstituted alkyl and most preferably methyl. However, it is most preferred that R₁ is hydrogen, i.e., n is zero.

n₁ is preferably zero or 1 and most preferably zero. The most preferred values of R₂ is hydrogen or lower alkyl, e.g., methyl. It is most preferred that R₂ is hydrogen.

It is preferred that n₂ is zero or 1. The most preferred value of R₃ is hydrogen, halo, or lower alkyl which is unsubstituted or substituted with halo. It is more preferred that R₃ is hydrogen, methyl or bromo, chloro or fluoro.

It is preferred that Y contains at least one ring nitrogen atom. It is even more preferred that if Y is a heteroaryl, it is attached to the CH₂ group at the nitrogen ring atom. The preferred heteroaryls are the specific heteroaryls described hereinabove with the more preferred heteroaryls being the most preferred heteroaryls described hereinabove.

Preferred compounds of formula I have the formula

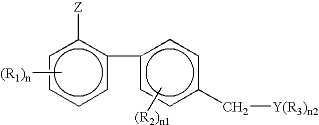

wherein R₁, Z, R₂, Y, R₃, n, n₁ and n₂ are as defined hereinabove.

An even more preferred embodiment of formula I has the formula

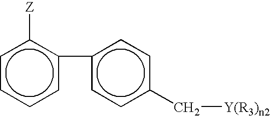

wherein Z, Y, R₃ and n₂ are as defined herein.

Even more preferred embodiments of formula I are

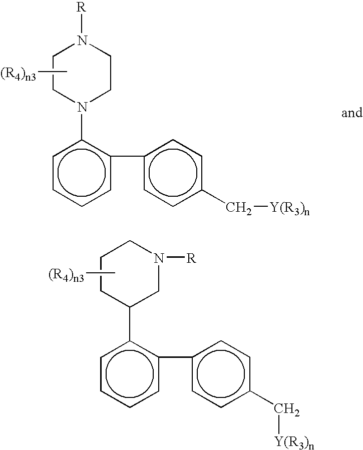

wherein R₄, R, R₃, Y, n and n₃ are as defined herein.

Preferred compounds of the present invention are

1-[2′-(4-Methyl-piperizine-1-yl)-biphenyl-4-ylmethyl]-1H-benzoimidazole;

5-Chloro-1-[2′-(4-methyl-piperazin-1-yl)-biphenyl-4-ylmethyl]-1H-benzoimidazole;

6-Chloro-1-[2′-(4-methyl-piperazin-1-yl)-biphenyl-4-ylmethyl]-1H-benzoimidazole;

1-(4′-Imidazol-1-ylmethyl-biphenyl-2-yl)-4-methyl-piperazine;

1-[2′-(4-Methyl-piperazin-1-yl)-biphenyl-4-ylmethyl]-1H-indole;

5-Fluoro-1-(2′-piperazin-1-yl-biphenyl-4-ylmethyl)-1H-indole;

5-Bromo-1-[2′-(4-methyl-piperazin-1-yl)-biphenyl-4-ylmethyl]-1H-indole;

5-Methyl-1-[2′-(4-methyl-piperazin-1-yl)-biphenyl-4-ylmethyl]-1H-indole;

1-Methyl-4-(4′-pyrrol-1-ylmethyl-biphenyl-2-yl)-piperazine;

2-Methyl-1-[2′-(4-methyl-piperazin-1-yl)-biphenyl-4-ylmethyl]-1H-indole;

1-[2′-(4-Methyl-piperazin-1-yl)-biphenyl-4-ylmethyl]-1H-pyrrolo[2,3-b]pyridine;

2-Methyl-1-[2′-(4-methyl-piperazin-1-yl)-biphenyl-4-ylmethyl]-1H-benzoimidazole;

1-Methyl-4-(4′-[1,2,4]triazol-1-ylmethyl-biphenyl-2-yl)-piperazine;

3-(4′-[1,2,4]Triazol-1-ylmethyl-biphenyl-2-yl)-piperidine;

3-[4′-(2-Ethyl-pyrrol-1-ylmethyl)-biphenyl-2-yl]-piperidine;

3-(4′-Pyrazol-1-ylmethyl-biphenyl-2-yl)-piperidine;

3-(4′-Pyrrol-1-ylmethyl-biphenyl-2-yl)-piperidine;

1-(2′-Piperidin-3-yl-biphenyl-4-ylmethyl)-1H-indole;

1-{4-[2-(4-Methyl-piperazin-1-yl)-pyridin-3-yl]-benzyl}-1H-benzoimidazole;

5-Chloro-1-{4-[2-(4-methyl-piperazin-1-yl)-pyridin-3-yl]-benzyl}-1H-benzoimidazole;

6-Chloro-1-{4-[2-(4-methyl-piperazin-1-yl)-pyridin-3-yl]-benzyl}-1H-benzoimidazole;

1-[3-(4-Imidazol-1-ylmethyl-phenyl)-pyridin-2-yl]-4-methyl-piperazine;

1-{4-[2-(4-Methyl-piperazin-1-yl)-pyridin-3-yl]-benzyl}-1H-indole;

5-Fluoro-1-{4-[2-(4-methyl-piperazin-1-yl)-pyridin-3-yl]-benzyl}-1H-indole;

5-Bromo-1-{4-[2-(4-methyl-piperazin-1-yl)-pyridin-3-yl]-benzyl}-1H-indole;

5-Methyl-1-{4-[2-(4-methyl-piperazin-1-yl)-pyridin-3-yl]-benzyl}-1H-indole;

1-Methyl-4-[3-(4-pyrrol-1-ylmethyl-phenyl)-pyridin-2-yl]-piperazine;

2-Methyl-1-{4-[2-(4-methyl-piperazin-1-yl)-pyridin-3-yl]-benzyl}-1H-indole;

1-{4-[2-(4-Methyl-piperazin-1-yl)-pyridin-3-yl]-benzyl}-1H-pyrrolo[2,3-b]pyridine;

2-Methyl-1-{4-[2-(4-methyl-piperazin-1-yl)-pyridin-3-yl]-benzyl}-1H-benzoimidazole;

and 1-Methyl-4-[3-(4-[1,2,4]triazol-1-ylmethyl-phenyl)-pyridin-2-yl]-piperazine;

and pharmaceutically acceptable salts thereof.

The compounds of formula I are prepared by art recognized techniques. For example an exemplary scheme is as follows:

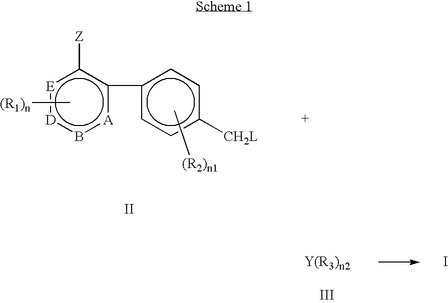

wherein R₁, Z, E, D, B, A R₂, n, Y, R₃ and R₂ is as defined herein and L is a leaving group known in the art. Examples of leaving groups include halo, and arylsulfonates such as brosyl, tosyl, mesyl, and nosyl, or trifluoroalkyl sulfonates, such as triflates or treslates or nonafluoroalkyl sulfonates, such as nonaflates.

A compound of formula II is reacted with a compound of formula III under substitution reaction conditions in the presence of a base, for example, KOH or NaOH, LiOH, alkali carbonate or trialkyl ammonium hydroxide, and the like in combination with a tetrabutylammonium salt, such as tetrabutylammonium hydrogen sulfate and the like. It is preferred that the base is NaOH, and that this reaction is conducted in a solvent which will dissolve the compounds of formula II and III, such as for example in a mixed solvent, e.g., toluene/H₂O, xylene or other hydrocarbon solvents, and the like. The preferred solvent is toluene/H₂O.

The reaction is performed under conditions effective to form the desired product. For example, the reaction may be effected at temperatures ranging from room temperature up to the reflux temperature of the solvent. It is preferred that the reaction is performed with slight heating, such as from about 30° C. to about 80° C. and more preferably from about 30° C. to about 60° C.

Alternatively, the product can be prepared in accordance with the procedure outlined in the following scheme

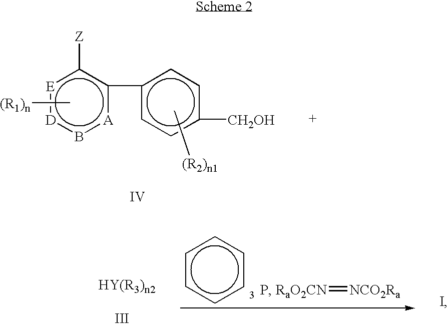

where R, R₁, Z, E, D, B, A, R₂, R₃, no, n₁, n₂, n₃, n₄ and Y are defined hereinabove. The reaction is effected by reacting the heteroaryl III with the alcohol IV with triphenylphosphine and RₐO₂CN═NCO₂Rₐ where Rₐ is lower alkyl, such as methyl or ethyl, e.g., diethyl azodicarboxylate, under effective reaction conditions. See, e.g., the article by Mitsonobu in Synthesis, 1981, 1, the contents of which are incorporated herein by reference. The reaction is preferably conducted in a solvent in which both III and IV are soluble, e.g., THF, ethers or halocarbon solvents, but preferably THF. Moreover, the reaction is conducted at effective temperatures, for example, at a temperature ranging from 20° C. to the reflux temperature, although it is preferred that the reaction is run at about 50° C.

Compounds of formula II and IV are prepared by art recognized techniques. An exemplary procedure is illustrated hereinbelow.

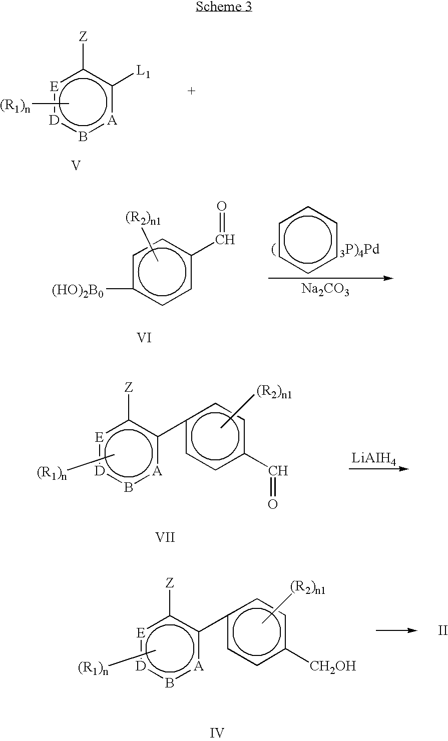

wherein R₁, R₂, Z, E, D, B and A, and n₁ are as defined hereinabove and L₁ is halide.

A compound of Formula V is reacted with 4-formylphenylboronic acid (Bo is boron) of formula VI in the presence of a base, such as sodium carbonate and tetrakis (triphenylphosphine) palladium under effective reaction conditions to form the aldehyde VII. The reaction is conducted in an inert gas, such as nitrogen or helium, and the like. Preferably, the reaction is conducted in a dry box under a nitrogen atmosphere. The reaction is preferably effected in a solvent in which V and VI are soluble, such as a mixed solvent, e.g., ethanol/water, however, before conducting the reaction, the solvent is preferably purged of oxygen, such as by under a stream of nitrogen. The reaction is conducted at elevated temperatures, e.g., from about 20° C. to the boiling point of the solvent and more preferably from about 80° C. to about 100° C.

The resulting aldehyde VII is reduced to the corresponding alcohol IV under reducing conditions such as using NaBH₄ or LiAlH₄. IV may be reacted with a compound of formula III as described hereinabove. Alternatively, IV is converted to II by standard reactions known in the art, such as reacting II with HL wherein L is as defined hereinabove. In addition, IV can be converted to the corresponding halide, e.g., chloride by standard techniques known in the art, for example using thionyl chloride, PCl₅, PCl₃, POCl₃, and the like.

A variation of the above scheme is depicted in Scheme IV

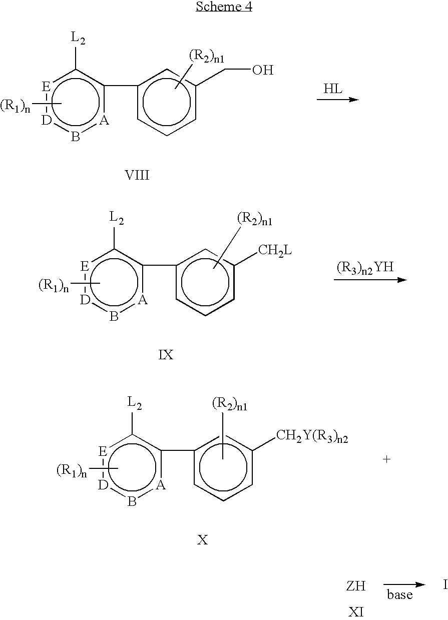

In an alternative scheme, the alcohol VII is reacted with HL under substitution reaction conditions, as described hereinabove. In the reaction scheme, L₂ is halide, and E, D, B, A, L, R₃, Y, R₁, n₁, n₂, R₂, n, and Z are as defined hereinabove and Y is a nitrogen containing heteroaryl which in the compound of formula I is attached to the CH₂ bridging group. IX is reacted with a compound of formula III under conditions described hereinabove in Scheme I. The product X, is reacted with ZH under conditions effective to form a compound of formula I. If Z is attached to the

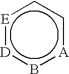

ring through a nitrogen ring atom, then it is reacted with ZH in the presence of a strong base, such as sodium t-butoxide, 2,2-bisdiphenylphosphanyl-[1,1]-binaphthalenyl and palladium salt, such as palladium (II) acetate under effective conditions to form 1. See, for example, the article by Wolfe, et al., in Acc. Chem. Research, 1998, 31, 805-818, the contents of which are incorporated by reference. The reaction is conducted in an inert solvent in which the reactants are soluble such as toluene. Although the reaction may be effected at temperatures ranging from about room temperature to the reflux temperature of the solvent, it is preferred that the reaction mixture is heated at a temperature ranging from about 20° C. to about 120° C. and more preferably form about 75° C. to about 120° C.

Although not shown, if Z is bonded through a nitrogen ring atom to

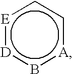

then the product X can be prepared from VIII by following the procedure in Scheme II.

On the other hand, if Z is attached to the

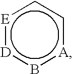

ring through a carbon ring atom, it is prepared by art recognized techniques. For example, by compound X is reacted with B(Et)₂ Z₁ wherein Z₁ is a heteroaryl in which the B is attached to the carbon atom, in the presence of a base under effective conditions to form a product

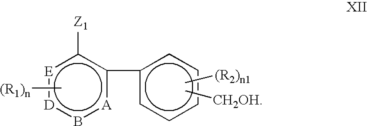

The reaction is conducted preferably in a solvent in which the reactants are soluble, such as a mixed solvent, e.g., THF/water. The reaction is conducted at an effective temperature which ranges from about room temperature to about 110° C. but preferably it is conducted at a temperature ranging from about 70° C. to about 90° C. The resulting product is reduced with effective reducing agents known in the art, such as lithium triethylborohydride in a solvent in which XII is soluble, e.g., THF or other ether solvents, wherein THF is preferred to produce a compound of formula IV which is then reacted with a compound of formula III, as described hereinabove to form a compound of formula I, which is converted to a compound of formula II.

In the reactions described hereinabove, if any of the substituents on the reactants are reactive under the reaction conditions then the substituent may be protected by protecting groups known in the art. Examples of such protecting groups can be found in a book entitled, Protective Groups in Organic Synthesis, by Theodora W. Greene, John Willey & Sons, NY, N.Y. 1981. Alternatively, the reactive substituents could be added to the product after completion of the reaction in which the substituent is reactive.

The compounds of formula I above may contain chiral centers and therefore exist in different enontiomeric forms. This invention relates to all optical isomers and all other stereoisomers of compounds of formula I and mixtures thereof.

The pharmaceutically acceptable salts include pharmaceutically acceptable acid addition salts of compound of formula I. The compounds of formula I are basic in nature and are capable of forming a wide variety of salts with various inorganic acids.

The acids that may be used to prepare pharmaceutically acceptable acid addition salts of those compounds of formula I are those that form non-toxic acid addition salts, i.e., salts containing pharmacologically acceptable ions, such as the hydrochloride, nitrate, sulfate, bisulfate, phosphate, citrate acid citrate, tartrate, pantothenate, butartrate, ascorbate, succinate, maleate, furmarate, glyconate, glucaronate, saccharate, formate, benzoate, glyconate, methane sulfonate, ethane sulfonate, benzene sulfonate and p-toluene sulfonate.

The present invention also includes isotopically labeled compounds, which are identical to those recited in formula I, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as ²H, ³H, ³C, ¹¹C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically labeled compounds of the present invention, for example those into which radioactive isotopes such as ³H and ¹⁴C are incorporated, are useful in drug and/or substrate tissue distribution assays. Tritiated, i.e., H, and carbon-14, i.e., C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of formula I of this invention and prodrugs thereof can generally be prepared by carrying out the procedures disclosed in the Schemes and/or in the Examples below, by substituting a readily available isotopically labeled reagent for a non-isotopically labeled reagent.

As indicated hereinabove, the present invention is also directed to a method of treating diseases or conditions caused by disorders of the serontonin system which comprises administering to a mammal in need of such treatment a therapeutically effective amount of formula I.

As used herein, the term “mammals” refers to a species of the class of Mammalia having mamminary glands and hair. Examples include dog, cat, cow, mule, horse, rabbit, monkey, sheep, human and the like. The preferred mammal is human.

The term “treating” as used herein, refers to retarding or reversing the progress of or alleviating or preventing either the disease, disorder or condition or one or more symptoms of such disorder or condition. The term “treatment” as used herein refers to the act of treating a disorder or condition, as the term “treating” is defined above.

The terms “disease” and “condition” unless otherwise indicated, encompass both chronic disease and conditions as well as diseases and conditions that are temporary in nature. A disease or condition treatable according to the invention can be one of sudden onset. A disease or condition covered by the present invention can be genetic or environmental in origin.

The term “disorder of the serotonin system” as referred to herein, refers to disorders,\ the treatment of which can be effected or facilitated by altering (i.e., increasing or decreasing) serotonin mediated neurotransmission.

The present invention also relates to a pharmaceutical composition for treating a disorder or condition selected from depression, anxiety, avoidant personality disorder, premature ejaculation, eating disorders (e.g., anorexia nervosa and bulimia nervosa), migraine, premenstrual syndrome, premenstrual dysphoric disorder, seasonal affective disorder, bipolar disorder, jet lag, sleep disorders such as circadian sleep rhythms disorder, sleep deprivation, REM sleep disorders, hypersomnia, parasomnias, sleep-wake cycle disorders, narcolepsy, sleep disorders associated with blindness, sleep disorders associated with obesity, and sleep disorders associated with shift work or irregular work schedules; nocturnal enuresis, and restless leg syndrome in a mammal, preferably a human, comprising an amount of a compound of formula I, or a pharmaceutically acceptable salt thereof, that is effective in treating such disorder or condition, and a pharmaceutically acceptable carrier.

The present invention also relates to a method for treating a disorder or condition selected from depression, anxiety, avoidant personality disorder, premature ejaculation, eating disorders (e.g., anorexia nervosa and bulimia nervosa), migraine, premenstrual syndrome, premenstrual dysphoric disorder, seasonal affective disorder, bipolar disorder, jet lag, sleep disorders such as circadian sleep rhythms disorder, sleep deprivation, REM sleep disorders, hypersomnia, parasomnias, sleep-wake cycle disorders, sleep disorders associated with blindness, sleep disorders associated with obesity, narcolepsy and sleep disorders associated with shift work or irregular work schedules; nocturnal enuresis, and restless leg syndrome in a mammal, preferably a human, comprising administering to a mammal, preferably a human, in need of such treatment an amount of a compound of the formula I, or a pharmaceutically acceptable salt thereof, that is effective in treating such disorder or condition.

The present invention also relates to a pharmaceutical composition for treating a disorder or condition selected from depression, anxiety, avoidant personality disorder, premature ejaculation, eating disorders (e.g., anorexia nervosa and bulimia nervosa), migraine, premenstrual syndrome, premenstrual dysphoric disorder, seasonal affective disorder, bipolar disorder, jet lag, sleep disorders such as circadian sleep rhythms disorder, sleep deprivation, REM sleep disorders, hypersomnia, parasomnias, sleep-wake cycle disorders, sleep disorders associated with blindness, sleep disorders associated with obesity, narcolepsy and sleep disorders associated with shift work or irregular work schedules; nocturnal enuresis, and restless leg syndrome in a mammal, preferably a human, comprising a 5HT7 receptor antagonizing (inverse agonizing) effective amount of a compound of the formula I, or a pharmaceutically acceptable salt thereof, and a pharmaceutically acceptable carrier.

The present invention also relates to a method for treating a disorder or condition selected from depression, anxiety, avoidant personality disorder, premature ejaculation, eating disorders (e.g., anorexia nervosa and bulimia nervosa), migraine, premenstrual syndrome, premenstrual dysphoric disorder, seasonal affective disorder, bipolar disorder, jet lag, sleep disorders such as circadian sleep rhythms disorder, sleep deprivation, REM sleep disorders, hypersomnia, parasomnias, sleep-wake cycle disorders, sleep disorders associated with blindness, sleep disorders associated with obesity, narcolepsy and sleep disorders associated with shift work or irregular work schedules; nocturnal enuresis, and restlessleg syndrome in a mammal, preferably a human, comprising administering to a mammal, preferably a human, requiring such treatment a 5HT7 receptor antagonizing or inverse agonizing effective amount of a compound of formula I or a pharmaceutically acceptable salt thereof.

The present invention also relates to a pharmaceutical composition for the treatment of a disorder or condition selected from depression, anxiety, avoidant personality disorder, premature ejaculation, eating disorders (e.g., anorexia nervosa and bulimia nervosa), migraine, premenstrual syndrome, premenstrual dysphoric disorder, seasonal affective disorder, bipolar disorder, jet lag, sleep disorders such as circadian sleep rhythms disorder, sleep deprivation, REM sleep disorders, hypersomnia, parasomnias, sleep-wake cycle disorders, sleep disorders associated with blindness, sleep disorders associated with obesity, narcolepsy and sleep disorders associated with shift work or irregular work schedules; nocturnal enuresis, and restless leg syndrome in a mammal, preferably a human, comprising: (a) an NK1 receptor antagonist or a pharmaceutically acceptable salt thereof; (b) a compound of formula I or a pharmaceutically acceptable salt thereof; and (c) a pharmaceutically acceptable carrier; wherein the NK1 receptor antagonist or pharmaceutical acceptable salt thereof and the compound of formula I or pharmaceutically acceptable salt thereof as described herein are together present in amounts that render the composition effective in treating such disorder or condition.

This invention also relates to a method of treating a disorder or condition selected from depression, anxiety, avoidant personality disorder, premature ejaculation, eating disorders (e.g., anorexia nervosa and bulimia nervosa), migraine, premenstrual syndrome, premenstrual dysphoric disorder, seasonal affective disorder, bipolar disorder, jet lag, sleep disorders such as circadian sleep rhythms disorder, sleep deprivation, REM sleep disorders, hypersomnia, parasomnias, sleep-wake cycle disorders, sleep disorders associated with blindness, sleep disorders associated with obesity, narcolepsy and sleep disorders associated with shift work or irregular work schedules; nocturnal enuresis, and restless leg syndrome in a mammal, preferably a human, comprising administering to said mammal, (a) an NK1 receptor antagonist or a pharmaceutically acceptable salt thereof; and (b) a compound of formula I or pharmaceutically acceptable salt thereof; wherein together the NK1 receptor antagonist or pharmaceutically acceptable salt thereof and the compound of formula I or pharmaceutically acceptable salt thereof are together present in amounts that render the combination effective in treating such disorder or condition.

The present invention also relates to a pharmaceutical composition for the treatment of a disorder or condition selected from depression, anxiety, avoidant personality disorder, premature ejaculation, eating disorders (e.g., anorexia nervosa and bulimia nervosa), migraine, premenstrual syndrome, premenstrual dysphoric disorder, seasonal affective disorder, bipolar disorder, jet lag, sleep disorders such as circadian sleep rhythms disorder, sleep deprivation, REM sleep disorders, hypersomnia, parasomnias, sleep-wake cycle disorders, sleep disorders associated with blindness, sleep disorders associated with obesity, narcolepsy and sleep disorders associated with shift work or irregular work schedules; nocturnal enuresis, and restless leg syndrome in a mammal, preferably a human, comprising: (a) a serotinon reuptake inhibitor, preferably sertraline, or a pharmaceutically acceptable salt thereof; (b) a compound of formula I or pharmaceutically acceptable salt thereof; and (c) a pharmaceutically acceptable carrier; wherein together the compound of formula I or pharmaceutically acceptable salt thereof and the serotinon reuptake inhibitor or pharmaceutically acceptable salt thereof are present in amounts that render the composition effective in treating such disorder or condition.

This invention also relates to a method of treating a disorder or condition selected from depression, anxiety, avoidant personality disorder, premature ejaculation, eating disorders (e.g., anorexia nervosa and bulimia nervosa), migraine, premenstrual syndrome, premenstrual dysphoric disorder, seasonal affective disorder, bipolar disorder, jet lag, sleep disorders such as circadian sleep rhythms disorder, sleep deprivation, REM sleep disorders, hypersomnia, parasomnias, sleep-wake cycle disorders, sleep disorders associated with blindness, sleep disorders associated with obesity, narcolepsy and sleep disorders associated with shift work or irregular work schedules; nocturnal enuresis, and restless leg syndrome in a mammal, preferably a human, comprising administering to said mammal, (a) a serotonin reuptake inhibitor, preferably sertraline, or a pharmaceutically acceptable salt thereof; and (b) a compound of formula I or pharmaceutically acceptable salt thereof; wherein together the compound of formula I or pharmaceutically acceptable salt thereof and the serotonin reuptake inhibitor or pharmaceutically acceptable salt thereof are together present in amounts that render the combination effective in treating such disorder or condition.

The present invention also relates to a pharmaceutical composition for the treatment of a disorder or condition selected from depression, anxiety, avoidant personality disorder, premature ejaculation, eating disorders (e.g., anorexia nervosa and bulimia nervosa), migraine, premenstrual syndrome, premenstrual dysphoric disorder, seasonal affective disorder, bipolar disorder, jet lag, sleep disorders such as circadian sleep rhythms disorder, sleep deprivation, REM sleep disorders, hypersomnia, parasomnias, sleep-wake cycle disorders, sleep disorders associated with blindness, sleep disorders associated with obesity, narcolepsy and sleep disorders associated with shift work or irregular work schedules; nocturnal enuresis, and restless leg syndrome in a mammal, preferably a human, comprising: (a) a 5HT1B receptor antagonist or a pharmaceutically acceptable salt thereof; (b) a compound of formula I or pharmaceutically acceptable salt thereof; and (c) a pharmaceutically acceptable carrier; wherein the compound of formula I or pharmaceutically acceptable salt thereof and the 5HT1B receptor antagonist or a pharmaceutically acceptable salt thereof are together present in amounts that render the composition effective in treating such disorder or condition.

This invention also relates to a method of treating a disorder or condition selected from depression, anxiety, avoidant personality disorder, premature ejaculation, eating disorders (e.g., anorexia nervosa and bulimia nervosa), migraine, premenstrual syndrome, premenstrual dysphoric disorder, seasonal affective disorder, bipolar disorder, jet lag, sleep disorders such as circadian sleep rhythms disorder, sleep deprivation, REM sleep disorders, hypersomnia, parasomnias, sleep-wake cycle disorders, sleep disorders associated with blindness, sleep disorders associated with obesity, narcolepsy and sleep disorders associated with shift work or irregular work schedules; nocturnal enuresis, and restless leg syndrome in a mammal, preferably a human, comprising administering to said mammal (a) a 5HT1B receptor antagonist or a pharmaceutically acceptable salt thereof; and (b) a compound of the formula I or pharmaceutically acceptable salt thereof; wherein the 5HT1B receptor antagonist or pharmaceutically acceptable salt thereof and the compound of formula I or pharmaceutically acceptable salt thereof are together present in amounts that render the combination effective in treating such disorder or condition.

Compounds of formula I and their pharmaceutically acceptable salts (hereinafter also referred to, collectively, as “the active compounds of this invention”) are antagonists and/or inverse agonists of the 5HT7 receptor. The active compounds are useful in the treatment of depression, anxiety, avoidant personality disorder, premature ejaculation, eating disorders (e.g., anorexia nervosa and bulimia nervosa), migraine, premenstrual syndrome, premenstrual dysphoric disorder, seasonal affective disorder, bipolar disorder, jet lag, sleep disorders such as circadian sleep rhythms disorder, sleep deprivation, REM sleep disorders, hypersomnia, parasomnias, sleep-wake cycle disorders, sleep disorders associated with obesity, narcolepsy, sleep disorders associated with blindness, and sleep disorders associated with shift work or irregular work schedules; nocturnal enuresis, and restless leg syndrome.

The compounds of the present are useful for the treatment of depression. As used herein, the term depression includes major depressive disorder, single episode or recurrent major depressive episodes; recurrent depression; dysthymia, cyclothymia, depressive disorders not otherwise specified, seasonal affective disorder; and bipolar disorders, for example, bipolar I disorder, bipolar II disorder and bipolar disorder not otherwise specified.

Other mood disorders encompassed within the term “depression”, as used herein, include dysthymic disorder with early or late onset and with or without atypical features; dementia of the Alzheimer's type, with early or late onset, with depressed mood; vascular dementia with depressed mood; mood disorders induced by alcohol, amphetamines, cocaine, hallucinogens, inhalants, opioids, phencyclidine, sedatives, hypnotics, anxiolytics or other substances; schizoaffective disorder of the depressed type; and adjustment disorder with depressed mood.

Also, encompassed within the term “depression”, as used herein, are: depression in cancer patients, depression in Parkinson's patients, postmyocardial infarction depression, subsyndromal symptomatic depression, depression in infertile women, pediatric depression, child abuse induced depression, and post partum depression.

Major depression is characterized by feelings of intense sadness and despair, mental slowing and loss of concentration, pessimistic worry, agitation, and self-deprecation. Physical changes also occur, especially in severe or “melancholic” depression. These include insomnia or hypersomnia, anorexia and weight loss (or sometimes overeating), decreased energy and libido, and disruption of normal circadian rhythms of activity, body temperature, and many endocrine functions. These are also encompassed by the term depression.

The compounds of the present invention are also useful for the treatment of anxiety. As used herein, the term “anxiety” includes anxiety disorders, such as panic disorder with or without agoraphobia, agoraphobia without history of panic disorder, specific phobias, for example, specific animal phobias, social phobias, obsessive-compulsive disorder, stress disorders including post-traumatic stress disorder and acute stress disorder, and generalized anxiety disorders.

“Generalized anxiety” is typically defined as an extended period (e.g., at least six months) of excessive anxiety or worry with symptoms on most days of that period. The anxiety and worry is difficult to control and may be accompanied by restlessness, being easily fatigued, difficulty concentrating, irritability, muscle tension, and disturbed sleep.

“Panic disorder” is defined as the presence of recurrent panic attacks followed by at least one month of persistent concern about having another panic attack. A “panic attack” is a discrete period in which there is a sudden onset of intense apprehension, fearfulness or terror. During a panic attack, the individual may experience a variety of symptoms including palpitations, sweating, trembling, shortness of breath, chest pain, nausea and dizziness. Panic disorder may occur with or without agoraphobia.

“Phobias” includes agoraphobia, specific phobias and social phobias. “Agoraphobia” is characterized by an anxiety about being in places or situations from which escape might be difficult or embarrassing or in which help may not be available in the event of a panic attack. Agoraphobia may occur without history of a panic attack. A “specific phobia” is characterized by clinically significant anxiety provoked by feared object or situation. Specific phobias include the following subtypes: animal type, cued by animals or insects; natural environment type, cued by objects in the natural environment, for example storms, heights or water; blood-injection-injury type, cued by the sight of blood or an injury or by seeing or receiving an injection or other invasive medical procedure; situational type, cued by a specific situation such as public transportation, tunnels, bridges, elevators, flying, driving or enclosed spaces; and other type where fear is cued by other stimuli. Specific phobias may also be referred to as simple phobias. A “social phobia” is characterized by clinically significant anxiety provoked by exposure to certain types of social or performance circumstances. Social phobia may also be referred to as social anxiety disorder.

Other anxiety disorders encompassed within the term “anxiety” include anxiety disorders induced by alcohol, amphetamines, caffeine, cannabis, cocaine, hallucinogens, inhalants, phencychdine, sedatives, hypnotics, anxiolytics and other substances, and adjustment disorders with anxiety or with mixed anxiety and depression.

Anxiety may be present with or without other disorders such as depression in mixed anxiety and depressive disorders. The compositions of the present invention are therefore useful in the treatment of anxiety with or without accompanying depression.

The present invention also relates to a pharmaceutical composition for treating a disorder or condition that can be treated by modulating serotonergic neurotransmission in a mammal, preferably a human, comprising a 5HT7 receptor antagonizing or inverse agonizing (inverse agonizing) effective amount of a compound of the formula I, or a pharmaceutically acceptable salt thereof, and a pharmaceutically acceptable carrier.

The present invention also relates to a method for treating a disorder or condition that can be treated by modulating serotonergic neurotransmission in a mammal, preferably a human, comprising administering to a mammal requiring such treatment a 5HT7 receptor antagonizing or inverse agonizing effective amount of a compound of the formula I or a pharmaceutically acceptable salt thereof.

The present invention relates to a pharmaceutical composition for treating a condition or disorder that can be treated by modulating serotonergic neurotransmission in a mammal, preferably a human, comprising:

a) a pharmaceutically acceptable carrier;

b) a compound of the formula I or a pharmaceutically acceptable salt thereof; and

c) a serotonin (5HT) reuptake inhibitor, e.g., fluvoxamine, sertraline, fluoxetine or paroxetine, preferably sertraline, or a pharmaceutically acceptable salt thereof;

wherein the amounts of the active compounds (i.e., the compound of formula I and the 5HT reuptake inhibitor) are such that the composition is effective in treating such disorder or condition.

The present invention also relates to a method for treating a disorder or condition that can be treated by modulating serotonergic neurotransmission in a mammal, preferably a human, comprising administering to a mammal requiring such treatment:

a) a compound of the formula I, or a pharmaceutically acceptable salt thereof; and

b) a 5HT reuptake inhibitor, preferably sertraline, or a pharmaceutically acceptable salt thereof;

wherein the amounts of the active compounds (i.e., the compound of formula I and the 5HT reuptake inhibitor) are such that the combination is effective in treating such disorder or condition.

Sertraline, (1S-cis)-4-(3,4-dichlorophenyl)-1,2,3,4-tetrahydro-N-methyl-1-naphthalenamine, as used herein has the chemical formula C₁₇H₁₇NCl₂ and the following structural formula

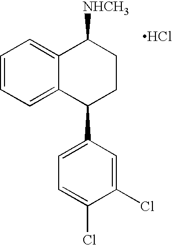

Its synthesis is described in U.S. Pat. No. 4,536,518, assigned to Pfizer Inc., the contents of which are incorporated herein by reference. Sertraline hydrochloride is useful as an antidepressant and anorectic agent, and is also useful in the treatment of depression, chemical dependencies, anxiety, obsessive compulsive disorders, phobias, panic disorder, post traumatic stress disorder, and premature ejaculation.

The present invention also relates to a method for treating a disorder or condition that can be treated by modulating serotonergic neurotransmission in a mammal, preferably a human, comprising administering to a mammal requiring such treatment:

a) a compound of formula I or a pharmaceutically acceptable salt thereof; and

b) a 5HT1B receptor antagonist or a pharmaceutically acceptable salt thereof;

wherein the amounts of the compound of formula I and the 5-HT1 B receptor antagonist taken together are such that the combination is effective in treating such disorder or condition.

The present invention also relates to a pharmaceutical composition for treating a disorder or condition that can be treated by modulating serotonergic neurotransmission in a mammal, preferably a human, comprising:

a) a pharmaceutically acceptable carrier.

b) a compound of formula I or a pharmaceutically acceptable salt thereof; and

c) a 5HT1B receptor antagonist or a pharmaceutically acceptable salt thereof;

wherein the amounts of the compound of formula I and the 5HT1B receptor antagonist taken together are such that the composition is effective in treating such disorder or condition.

The present invention also relates to a method for treating a disorder or condition that can be treated by modulating serotonergic neurotransmission in a mammal, preferably a human, comprising administering to a mammal requiring such treatment:

a) a compound of formula I or a pharmaceutically acceptable salt thereof; and

b) an NK1 receptor antagonist or a pharmaceutically acceptable salt thereof;

wherein the amounts of the compound of formula I and the NK1 receptor antagonist taken together are such that the combination is effective in treating such disorder or condition.

The present invention also relates to a pharmaceutical composition for treating a disorder or condition that can be treated by modulating serotonergic neurotransmission in a mammal, preferably a human, comprising:

a) a pharmaceutically acceptable carrier;

b) a compound of formula I or a pharmaceutically acceptable salt thereof; and

c) an NK1 receptor antagonist or a pharmaceutically acceptable salt thereof;

wherein the amounts of the compound of formula I and the NK1 receptor antagonist taken together are such that the composition is effective in treating such disorder or condition.

It will be appreciated that when using any of the combination methods of the present invention, referred to above, whichever components (a) and (b) that are utilized, i.e., whichever combination of a compound of formula I or pharmaceutically acceptable salt thereof and 5HT1B receptor antagonist or salt, NK1 receptor antagonist or salt or sertonin reuptake inhibitor or salt, the combination will be administered to a patient within a reasonable period of time. The compounds may be in the same pharmaceutically acceptable carrier and therefore administered simultaneously. They may be in separate pharmaceutical carriers such as conventional oral dosage forms that are taken simultaneously. The term combination, as used above, also refers to the case where the pharmaceutically active compounds are provided in separate dosage forms and are administered sequentially. Therefore, by way of example, the NK1 receptor antagonist may be administered as a tablet and then, within a reasonable period of time, the compound of the formula I may be administered either as an oral dosage form such as a tablet or a fast-dissolving oral dosage form. By a “fast dissolving oral formulation” is meant, an oral delivery form which when placed on the tongue of a patient, dissolves within about seconds.

and the pharmaceutically acceptable salts thereof.

Examples of NK1 receptor antagonists useful in this invention are the following compounds:

(2S,3S)-3-(6-methoxy-3-trifluoromethyl-1,3-dihydroisobenzofuran-5-yl)methylamino-2-phenylpiperidine;

(2S,3S)-3-(6-methoxy-1-methyl-1-trifluoromethylisochroman-7-yl)methylamino-2-phenylpiperidine;

(2S,3S)-3-(6-methoxy-3-methyl-3-trifluoromethyl-1,3-dihydroisobenzofuran-5-yl)methylamino-2-phenylpiperidine;

(2S,3S)-3-(6-methoxy-3-phenyl-3-trifluoromethyl-1,3-dihydroisobenzofuran-5-yl)methylamino-2-phenylpiperidine;

(2S,3S)-3-[1-(6-methoxy-3-methyl-3-trifluoromethyl-1,3-dihydroisobenzofuran-5-yl)ethylamino]-2-phenylpiperidine;

(2S,3S)-3-[(1R)-6-methoxy-1-methyl-1-trifluoromethylisochroman-7-yl]methylamino-2-phenylpiperidine;

(2S,3S)-3-[(3R)-6-methoxy-3-methyl-3-trifluoromethyl-1,3-dihydroisobenzofuran-5-yl)methylamino-2-phenylpiperidine;

(2S,3S)-N-(5-ethyl-2-methoxyphenyl)methyl-2-diphenylmethyl-1-azabi-cyclo[2.2.2]-octan-3-amine;

(2S,3S)-N-(5-isopropyl-2-methoxyphenyl)methyl-2-di-phenylmethyl-1-azabicyclo[2.2.2]-octan-3-amine;

(2S,3S)-N-(5-sec-butyl-2-methoxyphenyl)-methyl-2-diphenylmethyl-1-azabicyclo[2.2.2]-octan-3-amine;

(2S,3S)-N-(5-tert-butyl-2-methoxyphenyl)-methyl-2-diphenylmethyl-1-azabicyclo[2.2.2]-octan-3-amine; and

(2S,3S)-N-(5-methyl-2-methoxyphenyl)methyl-2-diphenylmethyl-1-azabicyclo[2.2.2]-octan-3-amine;

and the pharmaceutically acceptable salts thereof.

Other examples of this invention include the above combination methods wherein the NK1 receptor antagonist is a compound of the formula XIII selected from:

(2S,3S)-3-(5-tert-butyl-2-methoxybenzyl)amino-2-(3-trifluoromethoxyphenyl)piperidine;

(2S,3S)-3-(2-isopropoxy-5-trifluoromethoxybenzyl)amino-2-phenyl-piperidine;

(2S,3S)-3-(2-ethoxy-5-trifluoromethoxybenzyl)amino-2-phenyl-piperidine;

(2S,3S)-3-(2-methoxy-5-trifluoromethoxybenzyl)-amino-2-phenylpiperidine;

(2S,3S)-3(−5-tert-butyl-2-trifluoromethoxybenzyl)amino-2-phenylpiperidine;

2-(diphenylmethyl)-N-(2-methoxy-5-trifluoromethoxy-phenyl)methyl-1-azabicyclo[2.2.2]octan-3-amine;

(2S,3S)-3-[5-chloro-2-(2,2,2-trifluoroethoxy)-benzyl]amino-2-phenylpiperidine;

(2S,3S)-3-(5-tert-butyl-2-trifluoromethoxybenzyl)amino-2-phenylpiperidine;

(2S,3S)-3-(2-isopropoxy-5-trifluoromethoxybenzyl)amino-2-phenylpiperidine;

(2S,3S)-3-(2-difluoromethoxy-5-trifluoromethoxybenzyl)-amino-2-phenylpiperidine;

(2S,3S)-2-phenyl-3-[2-(2,2,2-trifluoroethoxybenzyl)-aminopiperidine; and

(2S,3S)-2-phenyl-3-(2-trifluoromethoxybenzyl)]aminopiperidine;

and pharmaceutically acceptable salts thereof.

Other embodiments of the present invention relate to the above combination methods wherein the NK1 receptor antagonist that is employed in such methods is selected from:

(2S,3S)-3-(6-methoxy-1-methyl-1-trifluoromethylisochroman-7-yl)methylamino-2-phenylpiperidine;

(2S,3S)-3-[(1R)-6-methoxy-1-methyl-1-trifluoromethylisochroman-7-yl]methylamino-2-phenylpiperidine;

(2S,3S)-N-(5-isopropyl-2-methoxyphenyl)methyl-2-di-phenylmethyl-1-azabicyclo[2.2.2]-octan-3-amine; and

(2S,3S)-N-(5-tert-butyl-2-methoxyphenyl)-methyl-2-diphenylmethyl-1-azabicyclo[2.2.2]-octan-3-amine;

and their pharmaceutically acceptable salts.

Examples of 5HT1 B antagonists that can be used in the pharmaceutical compositions and methods of this invention are the following:

3-(4-chlorophenyl)-5-[2-(4-methylpiperazin-1-yl)-benzylidene]-imidazolidine-2,4-dione;

3-(4-chlorobenzyl)-5-[2-(4-methylpiperazin-1-yl)-benzylidene]-imidazolidine-2,4-dione;

3-(4-chlorobenzyl)-5-[2-(4-methylpiperazin-1-yl)-benzylidene]-thiazolidine-2,4-dione;

4-benzyl-2-[2-(4-methylpiperazin-1-yl)-benzylidene]-thiomorpholin-3-one;

4-(3,4-dichlorobenzyl)-2-[2-(4-methylpiperazin-1-yl)-benzylidene]-thiomorpholin-3-one;

3-(4-chlorophenyl)-5-[2-(4-methylpiperazin-1-yl)-benzylidene]-thiazolidine-2,4-dione;

3-(4-trifluoromethylphenyl)-5-[2-(4-methylpiperazin-1-yl)-benzyl idene]-thiazolidine-2,4-dione;

2-[2-(4-methylpiperazin-1-yl)-benzylidene]-4-(4-trifluoromethylphenyl)-thiomorpholin-3-one;

2-[2-(4-methylpiperazin-1-yl)-benzylidene]-thiomorpholin-3-one;

4-(3,4-dichlorophenyl)-2-[2-fluoro-6-(4-methylpiperazin-1-yl)-benzylidene]-thiomorpholin-3-one;

4-(3,4-dichlorophenyl)-2-[2-(4-methylpiperazin-1-yl)-benzylidene]-morpholin-3-one;

4-(3,4-dichlorophenyl)-2-[2-(4-methylpiperazin-1-yl)-benzylidene]-thiomorpholin-3-one;

4-(3,4-dichlorophenyl)-2-[2-(4-methylpiperazin-1-yl)-benzyl]-thiomorpholin-3-one;

4-methyl-2-[2-(4-methylpiperazin-1-yl)-benzylidene]-thiomorpholin-3-one; and

4-(3,4-dichlorophenyl)-2-(2-piperazin-1-ylbenzylidene)-thiomorpholin-3-one.

and the pharmaceutically acceptable salts of such compounds.

Examples of serotonin reuptake inhibitors that can be used in the methods and compositions of this invention include, but are not limited to, sertraline, fluoxetine and paroxetine.

“Modulating serotonergic neurotransmission,” as used herein, refers to increasing or improving, or decreasing or retarding the neuronal process whereby serotonin is released by a pre-synaptic cell upon excitation and crosses the synapse to stimulate or inhibit the post-synaptic cell.

Unless indicated to the contrary, when used herein the term “active compounds” and “active agents” are synonymous and are therefore interchangeable. This term refers to the compounds of formula I and their pharmaceutically acceptable salts either alone or in combination with one or more of the compounds selected from the group consisting of 5HT1B receptor antagonists, NK1 receptor antagonists, 5HT receptor antagonists or pharmaceutically acceptable salts of any of the compounds identified herein.

The following references refer, collectively, to quinuclidine, piperidine, ethylene diamine, pyrrolidine and azanorbornane derivatives and related compounds that exhibit activity as NK1 receptor antagonists and can be used, in combination with the 5HT7 receptor antagonists and inverse agonists of the formula I, in the pharmaceutical compositions and methods of this invention, and to methods of preparing the NK1 receptor antagonists: U.S. Pat. No. 5,162,339, which issued on Nov. 11, 1992; U.S. Pat. No. 5,232,929, which issued on Aug. 3, 1993; World Patent Application WO 92/20676, published Nov. 26, 1992; World Patent Application WO 93/00331, published Jan. 7, 1993; World Patent Application WO 92/21677, published Dec. 10, 1992; World Patent Application WO 93/00330, published Jan. 7, 1993; World Patent Application WO 93/06099, published Apr. 1, 1993; World Patent Application WO 93/10073, published May 27, 1993; World Patent Application WO 92/06079, published Apr. 16, 1992; World Patent Application WO 92/12151, published Jul. 23, 1992; World Patent Application WO 92/15585, published Sep. 17, 1992; World Patent Application WO 93/10073, published May 27, 1993; World Patent Application WO93/19064, published Sep. 30, 1993; World Patent Application WO 94/08997, published Apr. 28, 1994; World Patent Application WO 94/04496, published Mar. 3, 1994; World Patent Application WO 95/07908, published Mar. 3, 1995; World Patent Application WO 90/14088, published November, 29, 1990; PCT/IBO2/13939, filed Sep. 20, 2002; World Patent Application WO 94/20500, published Sep. 15, 1994; World Patent Application WO 94/13663, published Jun. 23, 1994; World Patent Application WO 95/16679, published Jun. 22, 1995; World Patent Application WO 97/08144, published Mar. 6, 1997; World Patent Application WO 97/03066, published Jan. 30, 1997; World Patent Application WO 99/25714, published May 27, 1999; United States Patent Application 988,653, filed Dec. 10, 1992; United States Patent Application 026,382, filed Mar. 4, 1993; United States Patent Application 123,306, filed Sep. 17, 1993, and United States Patent Application 072,629, filed Jun. 4, 1993. The foregoing patents and patent applications are incorporated herein by reference in their entirety.

NK-1 receptor antagonists of the formula XIII can be prepared as described in the following patents and patent applications, all of which are referred to above and incorporated herein by reference in their entirety: WO 93/00331, WO 92/21677, WO 92/15585, WO 92/01688, WO 93/06099, WO 91/18899, U.S. Pat. No. 5,162,339, and U.S. Pat. No. 5,232,929.

Other NK1 receptor antagonists that can be used, in conjunction with the 5HT7 antagonists and/or inverse agonists of formula I for the treatment of anxiety or depression in accordance with the methods and pharmaceutical compositions of the present invention are those compounds and pharmaceutically acceptable salts described in the following references: European Patent Application EP 499,313, published Aug. 19, 1992; European Patent Application EP 520,555, published Dec. 30, 1992; European Patent Application EP 522,808, published Jan. 13, 1993, European Patent Application EP 528,495, published Feb. 24, 1993, World Patent Application WO 93/14084, published Jul. 22, 1993, World Patent Application WO 93/01169, published Jan. 21, 1993, PCT Patent Application WO 93/01165, published Jan. 21, 1993, World Patent Application WO 93/01159, published Jan. 21, 1993, World Patent Application WO 92,20661, published Nov. 26, 1992; European Patent Application EP 517,589, published Dec. 12, 1992; European Patent Application EP 428,434, published May 22, 1991,; and European Patent Application EP 360,390, published Mar. 28, 1990. The foregoing patents and patent applications are incorporated herein by reference in their entirety.

This invention relates to methods of treating anxiety, depression, and the other disorders referred to above in which an active compound of this invention and an NK1 receptor antagonist, 5HT1B receptor antagonist, or serotonin reuptake inhibitor are administered together, as part of the same pharmaceutical composition, as well as to methods in which the two active agents are administered separately as part of an appropriate dose regimen designed to obtain the benefits of the combination therapy. The appropriate dose regimen, the amount of each dose of an active agent administered, and the specific intervals between doses of each active agent will depend upon the subject being treated, the specific active agent being administered and the nature and severity of the specific disorder or condition being treated. In general, the active compounds of this invention, when used as a single active agent or in combination with another active agent, will be administered to an adult human in an amount from about 0.01 to about 2000 mg, in single or divided doses, preferably from about 0.1 to about 1000 mg. Such compounds may be administered on a regimen of up to 6 times per day, preferably 1 to 4 times per day, especially 2 times per day and most especially once daily. Variations may nevertheless occur depending upon the species of animal being treated and its individual response to said medicament, as well as on the type of pharmaceutical formulation chosen and the time period and interval at which such administration is carried out. In some instances, dosage levels below the lower limit of the aforesaid range may be more than adequate, while in other cases still larger doses may be employed without causing any harmful side effect, provided that such larger doses are first divided into several small doses for administration throughout the day.

A proposed daily dose of a 5HT reuptake inhibitor, preferably sertraline, in the combination methods and compositions of this invention, for oral, parenteral or buccal administration to the average adult human for the treatment of the conditions referred to above, is from about 0.01 mg to about 2000 mg, preferably from about 0.1 mg to about 200 mg of the 5HT reuptake inhibitor per unit dose, which could be administered, for example, 1 to 4 times per day.

A proposed daily dose of a 5HT1B receptor antagonist in the combination methods and compositions of this invention, for oral, parenteral, rectal or buccal administration to the average adult human for the treatment of the conditions referred to above, is from about 0.01 mg to about 200 mg, preferably from about 0.1 mg to about 50 mg of the 5HT1B receptor antagonist per unit dose, which could be administered, for example, 1 to 4 times per day.

A proposed daily dose of an NK1 receptor antagonist in the combination methods and compositions, for oral, parenteral or buccal administration to the average adult human for the treatment of the conditions referred to above, is from about 0.01 mg to about 1500 mg, preferably from about 0.05 mg to about 500 mg of the NK1 receptor antagonist per unit dose which could be administered, for example, 1 to 4 times per day.

The active agents may be administered alone or in combination with pharmaceutically acceptable carriers or diluents by either of the routes previously indicated, and such administration may be carried out in single or multiple doses. More particularly, the therapeutic agents of this invention can be administered in a wide variety of different dosage forms, i.e., they may be combined with various pharmaceutically acceptable inert carriers in the form of tablets, capsules, lozenges, troches, hard candies, suppositories, aqueous suspensions, injectable solutions, elixirs, syrups, and the like. Such carriers include solid diluents or fillers, sterile aqueous media and various non-toxic organic solvents, etc. Moreover, oral pharmaceutical compositions can be suitably sweetened and/or flavored. In general, the therapeutic agents of this invention, when administered separately (i.e., not in the same pharmaceutical composition) are present in such dosage forms at concentration levels ranging from about 5.0% to about 70% by weight.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid).

For preparing solid compositions such as tablets, the principal active ingredient is mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a therapeutic agent, or a non-toxic pharmaceutically acceptable salt thereof. When referring to these preformulation compositions as homogeneous, it is meant that the therapeutic agent is dispersed evenly throughout the composition so that the composition may be readily subdivided into equally effective unit dosage forms such as tablets, pills and capsules. This solid preformulation composition is then subdivided into unit dosage forms of the type described above containing, typically, from 0.05 to about 500 mg of each of the therapeutic agents contained in the composition. The tablets or pills of the composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac acetyl alcohol and cellulose acetate.

For buccal administration, the composition may take the form of tablets or lozenges formulated in conventional manner.

The active agents may be formulated for parenteral administration by injection, including using conventional catheterization techniques or infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulating agents such as suspending, stabilizing and/or dispersing agents. Solutions of a therapeutic agent in either sesame or peanut oil or in aqueous propylene glycol may be employed. The aqueous solutions should be suitably buffered if necessary and the liquid diluent first rendered isotonic. These aqueous solutions are suitable for intravenous injection purposes. The oily solutions are suitable for intraarticular, intramuscular and subcutaneous injection purposes. The preparation of all these solutions under sterile conditions is readily accomplished by standard pharmaceutical techniques well known to those skilled in the art. Alternatively, the active ingredient may be in powder form for reconstitution with a suitable vehicle, e.g., sterile pyrogen-free water, before use.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the invention and a suitable powder base such as lactose or starch.

Aerosol formulations of the active compounds of this invention for treatment of the conditions referred to above in the average adult human are preferably arranged so that each metered dose or “puff” of aerosol contains 20 μg to 1000 μg of active compound. The overall daily dose with an aerosol will be within the range 100 μg to 10 mg. Administration may be several times daily, for example 2, 3, 4 or 8 times, giving for example, 1, 2 or 3 doses each time.

The compounds of formula I may advantageously be used in conjunction with one or more other therapeutic agents, for instance, different antidepressant agents such as tricyclic antidepressants (e.g., amitriptyline, dothiepin, doxepin, trimipramine, butripyline, clomipramine, desipramine, imipramine, iprindole, lofepramine, nortriptyline or protriptyline), or monoamine oxidase inhibitors (e.g., isocarboxazid, phenelzine or tranylcyclopramine), and/or with antiparkinsonian agents such as dopaminergic antiparkinsonian agents (e.g., levodopa, preferably in combination with a peripheral decarboxylase inhibitor e.g., benserazide or carbidopa, or with a dopamine agonist e.g., bromocriptine, lysuride or pergolide). It is to be understood that the present invention covers the use of a compound of general formula (I) or a physiologically acceptable salt or solvate thereof in combination with one or more other therapeutic agents.

The affinities of the active compounds for 5HT7 receptors can be determined using standard radioligand binding assays as described in the literature. The 5HT7 affinity can be measured using the following procedure.

³H-5-Carboxamidotryptamine (3H-5-CT) Binding to Rat 5HT7 Receptors:

Materials:

Brinkman Polytron Tissue Homogenizer

Phosphate Buffered Saline (GIBCO)

Capped Centrifuge Tubes

Centrifuge

50 mMTris HClBuffer, pH7.7 (SigmaT-4378)

EDTA (Sigma E-4884)

MgSO₄ (Sigma M-7506)

CaCl₂ (MCBCX156)

pargyline (SigmaP-8013)

ascorbicacid (Calbiochem1831)

5-HTcreatinine sulfate complex (Sigma H-7752)

³H-5CT (Amersham TRK.1038)

12×75 mm boroscilicate glass tubes

96 well V-bottom polypropylene plates (NUNC—442587)

Skatron 96 Well Harvester

Whatman GF/B Glass Fiber Filters (Brandel FP-105) presoaked in 0.3%

polyethylenimine (Sigma—P-3143)

Betaplate scintillation counter (Wallac/LKB)

Tissue Preparation

Cells expressing rat 5HT7 receptors are grown according to standard cell culture techniques. Cells are harvested by removing the media, rinsing the flasks out with phosphate buffered saline (PBS) and then allowed to sit for 2-3 minutes with PBS containing 2.5 mM EDTA. Cells are dislodged and poured into a centrifuge tube. Flasks are rinsed with PBS and added to the centrifuge tube. The cells are centrifuged for ten minutes at 40,000×g (20,000 rpm in a Sorvall SS34 rotor). The supernatant is discarded and at this point the remaining pellet is weighed and can be stored frozen (−20 degrees C.) until used in the binding assay. Pellets (fresh or frozen) are homogenized in 50 mM Tris HCl buffer (pH 7.4 at 4 degrees C.) using a Polytron homogenizer (setting 15,000 rpm) for ten seconds in a biologcial hood certified for use with human tissues. The homogenate is centrifuged for ten minutes at 40,000×g. The supernatant is discarded and the pellet resuspended with the Polytron in a fresh ice-cold 50 mM Tris HCl (pH 7.4 at 4 degrees) buffer and centrifuged again. The final pellet is resuspended in assay buffer (50 mM Tris HCl buffer (pH 7.7 at 25 degrees) containing 0.5 mM EDTA, 10 mM MgSO₄, 2 mM CaCl₂) for a final tissue concentration of 5-15 mg wet weight of original pellet per mL buffer (2× final concentration).

Receptor Binding

Incubation is initiated by the addition of tissue to V-bottom polypropylene plates (in triplicate). Incubation is at 25 degrees C. for 2 hours.

Each tube receives:

100 uL tissue suspension (5-15 mg/mL original wet weight), 50 uL ³H-5-CT**(0.4 nM final concentration), and 50 uL drug or buffer

**³H-5-CT is made up in assay buffer that contains 40 uM pargyline and 0.4% ascorbic acid (for final concentrations of 10 uM pargyline & 0.1% ascorbic acid).

Nonspecific binding is determined using 1 uM 5-HT creatinine sulfate. Incubation is ended by rapid filtration under vacuum through fire-treated Whatman GF/B glass fiber filters (presoaked in 0.3% PEI for two hours and dried) using a 96 well Skatron Harvester (3 sec prewet; 20 seconds wash; 15 seconds dry). Filters are put into LKB sample bags with 10 mL BetaScint. Radioactivity is quantified by liquid scintillation counting using a BetaPlate counter (LKB).

The percent inhibition of specific binding is calculated for each concentration of test compound. An IC₅₀ value (the concentration which inhibits 50% of the specific binding) is determined by linear regression of the concentration-response data (log concentration vs. logit percent values). Kᵢ values are calculated according to Cheng and Prusoff: Kᵢ=IC₅₀/(1+(L/Kd)), where L is the concentration of the radioligand used in the experiment and the Kd value is the dissociation constant for the radioligand determined in separate saturation experiments. Preferred compounds of the present invention exhibit Kᵢ values ranging from about 0.1 nM to about 50 nM.

The following assay can be used to evaluate the functional activity of compounds at 5HT7 receptors.

5-HT7 Receptor Mediated Adenylate Cyclase Activity

Materials

1.5 mL siliconized polypropylene microfuge tubes (Costar 3207)

12×75 mm boroscilicate glass tubes

Heated water bath

Glass-Teflon Homogenizer

Centrifuge

cells expressing rat 5-HT7 receptors

32P-ATP (30 Ci/mmol: NEG-003—New England Nuclear)

3H-cAMP (30 Ci/mmol: NET-275—New England Nuclear)

1. Cells are grown according to standard cell culture techniques. Cells are harvested by replacing the media with phosphate-buffered saline containing 2.5 mM EDTA. The cells are homogenized using a hand-held glass-teflon homogenizer. The homogenate is centrifuged at 35,000×g for 10 minutes at 4 degrees C. The pellet is resuspended in 100 mM HEPES buffer containing 1 mM EGTA (pH 7.5) to a final protein concentration of 40 microgram protein per tube.

2. The Reaction Mix is prepared so that the following agents will be at these final concentrations in tube: 4.0 mM MgCl₂, 0.5 m MATP, 1.0 m McAMP, 0.5 mM IBMX, 10 mM, phosphocreatine, 0.31 mg/mL creatine phosphokinase, and 100 uM GTP0.5-1 microcuries a-[³²P]-ATP per tube.

3. Incubation is initiated by the addition of tissue to siliconized microfuge tubes (in triplicate). Incubation is at 37° C. for 15 minutes.

Each tube receives:

20 uL tissue, 20 uL drug or buffer (at 5× final concentration), 20 uL 100 nM agonist or buffer (at 5× final concentration), and 40 uL Reaction Mix

4. Incubation is terminated by the addition of 100 uL 2% SDS, 1.3 mM cAMP, 45 mM ATP solution containing 40,000 dpm [³H]-cAMP to monitor the recovery of cAMP from the columns. The separation of [³²P]-ATP and [³²P]-CAMP is accomplished using the method of Salomon et al., Analytical Biochemistry 58: 541-548, 1974, which is incorporated herein by reference in its entirety. Radioactivity is quantified by liquid scintillation counting.

The maximal effect of agonists is defined in terms of the maximal effect of serotonin (5-HT). Antagonists are evaluated by their ability to inhibit 5HT-stimulated adenylate cyclase activity. IC₅₀ values are converted to apparent Ki values by the following equation: IC50/(1+([agonist]/EC₅₀ of agonist)).

Preferred compounds of the present invention exhibit adenylate cyclase activity ranging from about 60 to about 150%.

Activity of a combination of active compounds to produce an antidepressant effect and related pharmacological properties can be determined by methods (1)-(4) below, which are described in Koe, B. et al., Journal of Pharmacology and Experimental Therapeutics, 226 (3), 686-700 (1983), which is incorporated herein by reference in its entirety. Specifically, activity can be determined by studying (1) their ability to affect the efforts of mice to escape from a swim-tank (Porsolt mouse “behavior despair” test), (2) their ability to potentiate 5-hydroxytryptophan-induced behavioral symptoms in mice in vivo, (3) their ability to antagonize the serotonin-depleting activity of p-chloroamphetamine hydrochloride in rat brain in vivo, and (4) their ability to block the uptake of serotonin, norepinephrine and dopamine by synaptosomal rat brain cells in vitro. The ability of the active combination to counteract reserpine hypothermia in mice in vivo can be determined according to the methods described in U.S. Pat. No. 4,029,731, which is incorporated herein by reference in its entirety.

The following Examples illustrate the preparation of the compounds of the present invention. Melting points are uncorrected. NMR data are reported in parts per million and are referenced to the deuterium lock signal from the sample solvent (deuteriochloroform unless otherwise specified). Specific rotations were measured at room temperature using the sodium D line (589 nm). Commercial reagents were utilized without further purification. THF refers to tetrahydrofuran. DMF refers to N,N-dimethylformamide. Chromatography refers to column chromatography performed using 47-61 micron mesh silica gel and executed under nitrogen pressure (flash chromatography) conditions. Room or ambient temperature refers to 20-25° C. All non-aqueous reactions were run under a nitrogen atmosphere for convenience and to maximize yields. Concentration at reduced pressure means that a rotary evaporator was used.

The following Examples illustrate the present invention. It is to be understood, however, that the invention, as fully described herein and as recited in the claims, is not intended to be limited by the details of the following Examples.

### EXAMPLE 1

### 1-[2′-(4-Methyl-piperizine-1-yl)-biphenyl-4-ylmethyl]-1H-benzoimidazole

Step 1

2′-(4-Methyl-piperazine-1-yl)-biphenyl-4-carboxaldehyde

A mixture of 55 ml of water in 520 ml of ethanol was purged of oxygen under a stream of N₂. To the solution were added 1-(2-bromo-phenyl)-4-methyl-piperazine (7.5 g, 29 mmol; Eur. Pat. Appl. (1999): EP 99-302288 19990325), 4-formylphenylboronic acid (8.8 g, 59 mmol), sodium carbonate (6.3 g, 59 mmol), and tetrakis(triphenylphosphine)-palladium(0) (1.71 g, 1.5 mmol). The system was evacuated under house vacuum and flushed with nitrogen, two times. Under a nitrogen atmosphere, the reaction mixture was stirred and heated at 90° C. for 18 hours. The cooled mixture was filtered through diatomaceous earth and concentrated to yield 19 g of an oil. Purification by flash chromatography (40 micron mesh silica gel; elution with methanol:dichloromethane, 3:97 in volume) afforded a yellow foam (8.2 g, quantitative yield).

Mass spectrum: m/z 281 (m+1).

Step 2

[2′-(4-Methyl-piperazine-1-yl)-biphenyl-4-yl]-methanol

A 1M solution of lithium aluminum hydride in THF (44.5 ml, 45 mmol) was added dropwise to an ice bath cooled solution of the title compound from Example 1, Step 1 (5.0 g, 17.8 mmol) in 50 mL of THF. The reaction mixture was stirred and cooled for one hour after addition of lithium aluminum hydride was complete, and then stirred for two additional hours at room temperature. After returning to an ice water cooling bath, the reaction mixture was quenched by dropwise addition of 8 mL of a 1N aqueous solution of NaOH, warmed to room temperature, diluted with 75 mL of THF and dried with sodium sulfate. The resulting mixture was filtered through diatomaceous earth and concentrated to yield an oil. Purification by flash chromatography (40 micron mesh silica gel; elution with MeOH:dichloromethane, 6:94 in volume) afforded an oil (2.34 g, 47% yield).

Mass spectrum: m/z 283 (m+1). Tlc R_{f} (silica gel plates; elution with methanol: dichloromethane, 6:94 in volume; UV detection): 0.54:

Step 3

1-[2′-(4-Methyl-piperizine-1-yl)-biphenyl-4-ylmethyl]-1H-benzoimidazole

To a solution of the title compound from Step 2 (100 mg, 0.35 mmol) in 4 ml of THF were added benzoimidazole (21 mg, 0.18 mmol), triphenylphosphine (92 mg, 0.35 mmol) and diethyl azodicarboxylate (55 ul, 0.35 mmol). The resulting reaction mixture was stirred at room temperature for 18 hours and then concentrated to an oil. This residue was partitioned between 15 ml 1 N aqueous NaOH and 15 ml of dichloromethane. The layers were separated and the aqueous portion was extracted with two 15 ml portions of dichloromethane. The combined organic extracts were dried (Na₂SO₄) and concentrated to an oil. Purification by flash chromatography (40 micron mesh silica gel; elution with MeOH:dichloromethane, 4:96, in volume) afforded a colorless gum (28 mg, 41% yield).

Mass spectrum: m/z 360 (m+1). Tlc R_{f} (silica gel plates; elution with methanol: dichloromethane, 4:94 in volume; UV detection): 0.25. ¹³C NMR (125 MHz, CDCl₃) delta 151.1, 144.9, 144.1, 142.1, 135.0, 134.5, 132.0, 130.3, 129.4, 127.9, 123.8, 123.6, 123.1, 121.2, 119.1, 110.8, 55.3, 51.1, 49.1, 46.2 ppm.

### EXAMPLE 2

5-Chloro-1-[2′-(4-methyl-piperazin-1-yl)-biphenyl-4-ylmethyl]-1H-benzoimidazole and

6-Chloro-1-[2′-(4-methyl-piperazin-1-yl)-biphenyl-4-ylmethyl]-1H-benzoimidazole

Step 1

5-Chloro-1-[2′-(4-methyl-piperazin-1-yl)-biphenyl-4-ylmethyl]-1H-benzoimidazole and

6-Chloro-1-[2′-(4-methyl-piperazin-1-yl)-biphenyl-4-ylmethyl]-1H-benzoimidazole

The title compounds were prepared in an analogous fashion to those in Example 1, Step 3 utilizing the title compound from Example 1, Step 2 (1.52 g, 5.4 mmol), 5-chlorobenzoimidazole (412 mg, 2.7 mmol), triphenylphosphine (1.41 g, 5.4 mmol) and diethyl azodicarboxylate (850 ul, 5.4 mmol). A mixture of the isomers listed above (760 mg, 68% yield) was generated. A portion of the above material was subjected to HPLC preparative chromatography (Chiral Technologies Chiralcel column (20 um, 10 cm×50 cm); elution with heptane: ethanol: diethylamine, 65:35:0.025, in volume; UV detection (220 nm)) affording the isomers as indicated below.

5-Chloro-1-[2′-(4-methyl-piperazin-1-yl)-biphenyl-4-ylmethyl]-1H-benzoimidazole (20% yield from mixture): Retention time on preparative column: 60 min.

¹³C NMR (125 MHz, CD₃OD) delta 148.5, 142.8, 141.8, 134.9, 133.0, 132.5, 131.4, 130.5, 130.0, 129.2, 128.6, 127.4, 124.5, 119.0, 115.2, 115.0, 53.8, 50.8, 48.5, 42.5 ppm.

6-Chloro-1-[2′-(4-methyl-piperazin-1-yl)-biphenyl-4-ylmethyl]-1H-benzoimidazole (41% yield from mixture): Retention time on preparative column: 70 min. ¹³C NMR (125 MHz, CDCl₃) delta 150.5, 144.5, 143.2, 141.9, 135.0, 134.5, 133.6, 131.7, 130.1, 129.3, 129.1, 127.5, 123.5, 123.3, 121.8, 118.9, 110.7, 100.0, 55.4, 51.2, 49.5, 46.3 ppm.

### EXAMPLE 3

1-(4′-Imidazol-1-ylmethyl-biphenyl-2-yl)-4-methyl-piperazine

Step 1

1-(4′-Imidazol-1-ylmethyl-biphenyl-2-yl)-4-methyl-piperazine

The title compound was prepared in an analogous fashion to Example 1, Step 3 utilizing the title compound from Example 1, Step 2 (100 mg, 0.35 mmol), imidazole (12 mg, 0.18 mmol), triphenylphosphine (92 mg, 0.35 mmol) and diethyl azodicarboxylate (55 uL, 0.35 mmol). The resulting crude material was purified by flash chromatography (40 micron mesh silica gel, elution with methanol: dichloromethane, 6:94 in volume, to methanol: dichloromethane, 10:90 in volume) affording an oil (15 mg, 13% yield)

Mass spectrum: m/z 333 (m+1). ¹H NMR (400 MHz, CDCl₃) delta 7.6 (m,3H), 7.23 (m,2H), 7.17 (m, 2H), 7.06 (m,3H), 6.93 (s, 1H), 5.12 (s,2H), 2.87 (m, 4H), 2.36(br s, 3H), 2.29 (m, 4H) ppm.

### EXAMPLE 4

1-[2′-(4-Methyl-piperazin-1-yl)-biphenyl-4-ylmethyl]-1H-indole

Step 1

Methanesulfonic acid 2′-(4-methyl-piperazin-1-yl)-biphenyl-4-ylmethyl ester

To an ice bath cooled solution of the title compound from Example 1, Step 2 (200 mg, 0.71 mmol) in 5 ml dichloromethane were added triethylamine (124 ul, 0.89 mmol) and methanesulfonyl chloride (60 ul, 0.78 mmol). The reaction mixture was stirred for 15 minutes, then diluted with 10% aqueous sodium bicarbonate (15 ml) and extracted with three 15 ml portions of dichloromethane. The organic extracts were combined, dried (Na₂SO₄) and diluted with 1 ml of toluene. The dichloromethane was removed in vacuo and the resulting solution of the unstable title compound in toluene was used immediately without further isolation or purification in Step 2.

Step 2

1-[2′-(4-Methyl-piperazin-1-yl)-biphenyl-4-ylmethyl]-1H-indole

To a solution of sodium hydroxide (500 mg) in water (500 ul) were added indole (55 mg, 0.47 mmol), tetrabutylammonium hydrogensulfate (12 mg, 0.05 mmol) and a solution of the title compound from Example 4 Step 1 (assume yield quantitative, 0.71 mmol) in 1 ml of toluene. The reaction mixture was stirred and heated at 33° C. for four hours and stirred at room temperature for 14 hours. Following dilution with 5 ml of water, the reaction mixture was extracted with three 10 ml portions of dichloromethane, dried (Na₂SO₄), and concentrated to an oil. Purification by flash chromatography (40 micron mesh silica gel; elution with methanol: dichloromethane, 4:96 in volume) afforded the title compound as an oil (68 mg, 25% yield).

Mass spectrum: m/z 382 (m+1).

Tlc R_{f} (silica gel plates; elution with methanol: dichloromethane, 4:96 in volume; UV detection): 0.36

¹³C NMR (125 MHz, CDCl₃) delta 150.2, 140.5, 136.0, 135.8, 134.4, 131.3, 129.2, 128.8, 128.4, 128.3, 126.8, 122.7, 121.6, 121.0, 119.5, 118.3, 109.8, 101.6, 55.1, 50.9, 50.1, 46.1 ppm.

### EXAMPLE 5

5-Fluoro-1-(2′-piperazin-1-yl-biphenyl-4-ylmethyl)-1H-indole

Step 1

4-(2-Bromo-phenyl)-piperazine-1-carboxylic acid tert-butyl ester

Piperazine-1-carboxylic acid tert-butyl ester (10 g, 54 mmol) was added to a solution of 1,2-dibromobenzene (9.8 ml, 81 mmol) in toluene (150 ml). To this mixture were added 2,2′-bis-diphenylphosphanyl-[1,1]binaphthalenyl (672 mg, 1.1 mmol), palladium(II)acetate (248 mg, 1.1 mmol), and sodium-tert-butoxide (7.27 g, 76 mmol). The resulting mixture was heated at 120° C. for four hours and stirred at room temperature for 14 hours. The reaction mixture was then diluted with water (150 ml) and extracted with two 200 ml portions of ethyl acetate. The combined organic extracts were dried (Na₂SO₄) and concentrated to a black oil. Purification by flash chromatography (40 micron mesh silica gel; elution with ethyl acetate:hexanes, 10:90 in volume) afforded the title compound as an oil (9.4 g, 52% yield).

Mass spectrum: m/z 342 (m+1).

Tlc R_{f} (silica gel plates; elution with ethyl acetate:hexanes, 10:90 in volume; UV detection): 0.34.

¹³C NMR (125 MHz, CDCl₃) delta 154.9, 133.9, 128.3, 124.7, 121.0, 79.8, 51.6, 28.5 ppm.

Step 2

4-(4′-Formyl-biphenyl-2-yl)-piperazine-1-carboxylic acid tert-butyl ester

The title compound was prepared in an analogous fashion to Example 1, Step 1, utilizing the title compound from Example 5, Step 1 (9.4 g, 28 mmol), 4-formylphenylboronic acid (8.25 g, 55 mmol), sodium carbonate (5.82 g, 55 mmol), and tetrakis(triphenylphosphine)palladium(0) (1.61 g, 1.4 mmol). The crude product was purified by flash chromatography (40 micron mesh silica gel; elution with ethyl acetate:hexanes, 12:88 in volume) to afford the title compound as a yellow solid (5.7 g, 56% yield).

Tlc R_{f} (silica gel plates; elution with ethyl acetate:hexanes, 12:88 in volume; UV detection): 0.44.

¹³C NMR (125 MHz, CDCl₃) delta 192.2, 155.0, 150.4, 147.8, 135.1, 134.1, 131.5, 130.0, 129.8, 123.6, 119.0, 80.0, 51.4, 28.6 ppm.

Step 3

4-(4′-Hydroxymethyl-biphenyl-2-yl)-piperazine-1-carboxylic acid tert-butyl ester

A 1M solution of lithium aluminum hydride in THF (31.2 ml, 31.2 mmol) was added dropwise to an ice bath cooled solution of the title compound from Example 5, Step 2 in 40 ml of THF. The reaction mixture was stirred for 15 minutes after addition of lithium aluminum hydride was complete and then quenched by dropwise addition of 8 ml of 1N aqueous NaOH. After the quench was complete the reaction mixture was warmed to room temperature, diluted with 50 ml of THF, and dried with Na₂SO₄. The resulting mixture was filtered through diatomaceous earth and concentrated to yield an oily solid. Purification by flash chromatography (40 micron mesh silica gel; elution with ethyl acetate:hexanes, 30:70 in volume) afforded the title compound as an oily solid (3.53 g, 61% yield).

Mass spectrum: m/z 369 (m+1).

Tlc R_{f} (silica gel plates; elution with ethyl acetate:hexanes, 30:70 in volume; UV detection): 0.50.

¹H NMR (400 MHz, CDCl₃) delta 7.62 (m, 2H), 7.39 (m, 2H), 7.25 (m, 2H), 7.08 (m,1H), 7.00 (m,1H), 4.72(s, 2H), 3.29(m, 4H), 2.77(m, 4H), 1.42 (s, 9H) ppm.

Step 4

4-(4′-Methanesulfonyloxymethyl-biphenyl-2-yl)-piperazine-1-carboxylic acid tert-butyl ester

The title compound from Example 5, Step 3 (200 mg, 0.54 mmol) was dissolved in dichloromethane (2 ml) and chilled in an ice water bath. To this solution were added triethylamine (94 ul, 0.68 mmol) and methanesulfonyl chloride (46 ul, 0.6 mmol). The resulting mixture was stirred ten minutes, removed from the cooling bath and stirred 30 min at room temperature. Following dilution with 10% aqueous sodium bicarbonate solution (8 ml) the reaction mixture was extracted with three 15 ml portions of dichloromethane. The combined organic extracts were dried (Na₂SO₄) and concentrated afford the title compound as an oil. This material was used immediately without further purification in Example 5, Step 5. The yield was assumed to be quantitative.

Step 5

4-[4′-(5-Fluoro-indol-1-ylmethyl)-biphenyl-2-yl]-piperazine-1-carboxylic acid tert-butyl ester

To a solution of sodium hydroxide (380 mg) in water (400 ul) were added 5-fluoroindole (74 mg, 0.55 mmol), tetrabutylammonium hydrogensulfate (9.3 mg, 0.03 mmol) and a solution of the title compound from Example 5, Step 4 (0.54 mmol) in toluene (1.5 ml). The reaction mixture was heated at 33° C. for 18 hours, cooled, diluted with 10% aqueous sodium bicarbonate solution (5 ml), and extracted with three 8 ml portions of dichloromethane. The combined organic extracts were dried (Na₂SO₄) and concentrated to provide an oil. Purification by flash chromatography (40 micron mesh silica gel; elution with ethyl acetate:hexanes, 8:92 in volume) afforded a white foam (109 mg, 42% yield).

Mass spectrum: m/z 386 (m+1-BOC).

Tlc R_{f} (silica gel plates; elution with ethyl acetate:hexanes, 8:92; UV detection): 0.42.

¹³C NMR (125 MHz, CDCl₃) delta 159.3, 157.0, 155.1, 150.3, 140.7, 136.0, 134.9, 133.1, 131.6, 130.2, 129.6, 128.8, 126.9, 123.3, 118.8, 110.7, 110.6, 110.4, 110.2, 106.1, 105.8, 101.8, 101.7, 80.0, 51.2, 50.6, 28.7 ppm.

Step 6

5-Fluoro-1-(2′-piperazin-1-yl-biphenyl-4-ylmethyl)-1H-indole

To the title compound from Example 5, Step 5 (109 mg, 0.22 mmol) was added diethylether saturated with HCl gas (3 ml). The mixture was stirred at room temperature for 18 hours and then concentrated to a light pink solid HCl salt (94 mg, quantitative yield).

Mass spectrum: m/z 386 (m+1).

¹H NMR (400 MHz, CD₃OD) delta 7.88 (m, 1H), 7.52 (m, 3H), 7.38 (m,1H), 7.31 (m,2H), 7.21 (m, 2H), 7.12 (m, 2H), 5.40 (m, 2H), 2.98 (m, 8H) ppm.

### Example 6

5-Bromo-1-[2′-(4-methyl-piperazin-1-yl)-biphenyl-4-ylmethyl]-1H-indole

Step 1

4-[4′-(5-Bromo-indol-1-ylmethyl)-biphenyl-2-yl]-piperazine-1-carboxylic acid tert-butyl ester

The title compound was prepared in an analogous fashion to the title compound from Example 5, Step 5, utilizing NaOH (380 mg), H₂O (400 ul), 5-bromoindole (106 mg, 0.54 mmol), tetrabutylammonium hydrogensulfate (9.2 mg, 0.27 mmol) and the title compound form Example 5, Step 4 (0.54 mmol). The crude oil was purified by flash column chromatography (40 micron mesh silica gel; elution with ethyl acetate:hexanes, 12:88 in volume) to afford a colorless oil (196 mg, 66% yield)

Mass spectrum: m/z 446, 448 (m+1-BOC, m+2-BOC).

Tlc R_{f} (silica gel plates, elution with ethyl acetate:hexanes, 15:85, UV detection): 0.47.

¹³CNMR (125 MHz, CDCl₃) delta 155.5, 150.3, 140.7, 135.8, 134.8, 131.6, 130.7, 129.8, 129.6, 128.8, 126.9, 124.7, 123.7, 123.4, 118.7, 113.2, 111.5, 101.5, 80.0, 51.3, 50.4, 28.7 ppm.

Step 2

5-Bromo-1-(2′-piperazin-1-yl-biphenyl-4-ylmethyl)-1H-indole

To the title compound form Example 6, Step 1 (195 mg, 0.36 mmol) were added chloroform (10 ml) and diethyl ether saturated with HCl gas (10 ml). The mixture was stirred at room temperature for 18 hours and concentrated to a pink solid HCl salt (119 mg, 67% yield).

¹H NMR (400 MHz, CD₃OD) delta 7.69 (s, 1H), 7.51 (m, 3H), 7.20 (m, 7H), 5.41 (m, 2H), 2.97 (m, 8H) ppm.

Step 3

5-Bromo-1-[2′-(4-methyl-piperazin-1-yl)-biphenyl-4-ylmethyl]-1H-indole

To a solution of the title compound from Example 6, Step 2 (109 mg, 0.25 mmol) dissolved in 1.0 ml of THF were added formic acid (19 ul, 0.5 mmol) and a 37% aqueous formalin solution (22 ul, 0.3 mmol). The reaction was heated at 80° C. for four hours and then additional portions of formic acid (19 ul, 0.5 mmol) and a 37% aqueous formalin solution (22 ul, 0.3 mmol) were added. The resulting mixture was heated at 80° C. for one hour and stirred at room temperature for 17 hours. Following dilution with a 10% aqueous sodium bicarbonate solution (5 ml), the mixture was extracted with three 8 ml portions of dichloromethane, dried (Na₂SO₄), and concentrated to an oil. Purification by flash chromatography (40 micron mesh silica gel; elution with methanol: dichloromethane, 4:96 in volume) afforded pure material (3.5 mg, 3% yield)

Mass spectrum: m/z 462 (m+1).

¹HNMR (400 MHz, CDCl₃) delta 7.76 (m, 1H,), 7.35 (m, 2H), 7.15 (m, 8H), 6.49 (m, 1H), 5.32 (s, 2H), 2.79 (m, 4H), 2.23 (m, 7H) ppm.

### Example 7

5-Methyl-1-[2′-(4-methyl-piperazin-1-yl)-biphenyl-4-ylmethyl]-1H-indole

Step 1

Methanesulfonic acid 2′-bromo-biphenyl-4-ylmethyl ester

To an ice bath cooled solution of (2′-bromo-biphenyl-4-yl)-methanol (3.00 g, 11 mmol; PCT Int. Appl. (1997), WO 97-US5383 19970401) in dichloromethane (50 ml) were added triethylamine (1.98 ml, 14.2 mmol) and methanesulfonyl chloride (970 ul, 12.5 mmol). The resulting mixture was stirred for 10 minutes and then removed from the cooling bath and stirred 20 minutes. Additional portions of triethylamine (900 ul, 6.5 mmol) and methanesulfonyl chloride (485 ul, 6.3 mmol) were added and the mixture was stirred at room temperature for 18 hours. The reaction mixture was then diluted with 10% aqueous sodium bicarbonate (50 ml) and extracted with three 50 ml portions of dichloromethane. The combined organic extracts were dried (Na₂SO₄) and concentrated to an oil. Purification by flash chromatography (40 micron mesh silica gel; elution with ethyl acetate:hexanes, 5:95, in volume) afforded the title compound as an oil (2.71 g, 72% yield).

Tlc R_{f} (silica gel plates; elution with ethyl acetate:hexanes, 5:95 in volume, UV detection): 0.62.

¹H NMR (400 MHz, CDCl₃) delta 7.68 (m,1H), 7.60 (m, 3H), 7.43 (m, 3H), 7.24 (m, 1H), 4.64 (s, 3H) ppm.

Step 2

1-(2′-Bromo-biphenyl-4-ylmethyl)-5-methyl-1H-indole

To a solution of sodium hydroxide (541 mg) in water (600 ul) were added 5-methyl indole (101 mg, 0.77 mmol) tetrabutylammonium hydrogensulfate (13 mh, 0.03 mmol) and a solution of the title compound from Example 7, Step 1 (264 mg, 0.77 mmol) in toluene (2 ml) The resulting mixture was heated at 33° C. for 18 hours. After cooling, the mixture was diluted with 5 ml of water and extracted with three 15 ml portions of dichloromethane. The combined organic extracts were dried (Na₂SO₄) and concentrated to an oil. Purification by flash chromatography (40 micron mesh silica gel; elution with ethyl acetate:hexanes, 5:95 in volume (ethyl acetate was added to complete solution of crude material)) afforded product as an oil (159 mg, 55% yield).

Mass spectrum: m/z 377, 378 (m+1, m+2).

Tlc R_{f} (silica gel plates; elution with ethyl acetate:hexanes, 5:95 in volume; UV detection): 0.39.

¹H NMR (400 MHz, CDCl₃) delta 7.54 (m,4H), 7.27 (m, 8H), 7.04 (m, 1H), 6.51 (m, 1H), 5.34, (s,2H), 2.47 (s,3H) ppm.

Step 3

5-Methyl-1-[2′-(4-methyl-piperazin-1-yl)-biphenyl-4-ylmethyl]-1H-indole

To a solution of the title compound from Example 7, Step 2 (159 mg, 0.42 mmol) in toluene (5 ml) were added 1-methylpiperazine (283 ul, 2.5 mmol), palladium(II)acetate (9.6 mg, 0.042 mmol), 2,2′-bisdiphenylphosphanyl-[1,1′]binaphthalenyl (26 mg, 0.042 mmol), and sodium-tert-butoxide (121 mg, 1.26 mmol). The reaction mixture was heated at 120° C. for 18 hours and then additional portions of 1-methylpiperazine (283 ul, 2.5 mmol), palladium(II)acetate (9.6 mg, 0.042 mmol), 2,2′-bisdiphenylphosphanyl-[1,1′]binaphthalenyl (26 mg, 0.042 mmol), and sodium-tert-butoxide (121 mg, 1.26 mmol) were added. The resulting mixture was heated at 120° C. for seven hours and then stirred at room temperature for 18 hours. Following dilution with 15 ml of water, the mixture was extracted with three 15 ml portions of dichloromethane. The combined organic extracts were dried (Na₂SO₄) and concentrated to an oil. Purification by flash chromatography (40 micron mesh silica gel; elution with methanol:dichloromethane, 4:96 in volume) afforded the product as an oil (20.5 mg, 12% yield).

Mass spectrum: m/z 396 (m+1).

Tlc R_{f} (silica gel plates; elution with methanol: dichloromethane, 4:96 in volume; UV detection): 0.38.

¹³C NMR (125 MHz, CDCl₃) delta 140.4, 136.1, 134.5, 131.3, 129.2, 128.7, 128.4, 126.7, 123.2, 122.9, 120.6, 118.4, 112.5, 109.5, 101.0, 54.9, 50.5, 50.2, 45.7, 21.4 ppm.

### Example 8

1-Methyl-4-(4′-pyrrol-1-ylmethyl-biphenyl-2-yl)-piperazine

Step 1

1-(2′-Bromo-biphenyl-4-ylmethyl)-1H-pyrrole

The title compound was prepared in an analogous fashion to Example 7, Step 2, utilizing sodium hydroxide (1.78 g, 44 mmol), water (2 ml), tetrabutylammonium hydrogensulfate (50 mg, 0.148 mmol) and the title compound from Example 7, Step 1 (2.96 mmol) dissolved in 8 ml of toluene. Purification by flash column chromatography (40 micron mesh silica gel; elution with ethyl acetate:hexanes, 4:96 in volume) afforded the product as an oil (256 mg, 27% yield).

Mass spectrum: m/z 313 (m+1).

¹³C NMR (125 MHz, CDCl₃) delta 142.1, 140.5, 137.7, 133.2, 131.3, 129.8, 128.9, 127.5, 126.6, 122.6, 121.3, 108.7, 53.12 ppm.

Step 2

1-Methyl-4-(4′-pyrrol-1-ylmethyl-biphenyl-2-yl)-piperazine

The title compound was prepared in an analogous fashion to Example 7 Step 3, utilizing the title compound from Example 8, Step 1 (123 mg, 0.39 mmol) in 2 ml of toluene. 1-methylpiperazine (264 ul, 2.4 mmol), palladium(II)acetate (41 mg, 0.18 mmol), 2,2′-bisdiphenylphosphanyl-[1,1′]binaphthalenyl (112 mg, 0.18 mmol), and sodium-tert-butoxide (300 mg, 3.12 mmol). After heating at 33° C. for 18 hours, the reaction mixture was diluted with water (5 ml) and extracted with three 15 ml portions of dichloromethane. Purification by flash column chromatography (40 micron mesh silica gel; elution with methanol: dichloromethane; 4:96 by volume) afforded 45 mg of product as an oil.

Mass spectrum: m/z 332 (m+1).

¹³CNMR (125 MHz, CDCl₃) delta 150.5, 140.8, 136.7, 134.6, 131.6, 129.4, 128.7, 127.2, 122.9, 121.4, 118.6, 108.7, 55.4, 53.4, 51.2, 46.3 ppm.

### Example 9

2-Methyl-1-[2′-(4-methyl-piperazin-1-yl)-biphenyl 4-ylmethyl-1H-indole

Step 1

1-(2′-Bromo-biphenyl-4-ylmethyl)-2-methyl-1H-indole

The title compound was prepared in an analogous fashion to Example 7, Step 2 utilizing sodium hydroxide (667 mg), water (750 ul), 2-methylindole (125 mg, 0.95 mmol), tetrabutylammonium hydrogensulfate (16 mg, 0.05 mmol) and the title compound from Example 7, Step 1 (0.95 mmol) dissolved in 4 ml of toluene. Purification by flash column chromatography (40 micron mesh silica gel; elution with ethyl acetate:hexanes, 3:97 in volume) afforded the title compound as an oil (51 mg, 15% yield).

Mass spectrum: m/z 376, 378 (m, m+2).

Tlc R_{f} (silica gel plates; elution with ethyl acetate:hexanes, 3:97 in volume; UV detection): 0.43.

¹H NMR (400 MHz, CDCl₃) delta 7.64 (m, 1H), 7.57 (m, 1H), 7.3 (m, 5H), 7.11 (m, 5H), 6.35 (br s, 1H), 5.36, (s, 2H), 2.40 (s, 3H) ppm.

Step 2

2-Methyl-1-[2′-(4-methyl-piperazin-1-yl)-biphenyl-4-ylmethyl]-1H-indole

The title compound was prepared in an analogous fashion to Example 7, Step 3, utilizing the title compound from Example 9, Step 1 (51 mg, 0.14 mmol) in 0.75 ml toluene, 1-methyl piperazine (91 ul, 0.81 mmol), palladium(II)acetate (6.4 mg, 0.028 mmol), 2,2′-bisdiphenylphosphanyl-[1,1′]binaphthalenyl (17.4 mg, 0.028 mmol) and sodium-tert-butoxide (108 mg, 1.1 mmol). The reaction mixture was heated at 120° C. for six hours and stirred at room temperature for 18 hours. The reaction mixture was worked up analogously to Example 7, Step 3, and purification by flash chromatography (40 micron mesh silica gel; elution with methanol: dichloromethane, 5:95 in volume) afforded the title compound as an oil (15 mg, 27% yield).

Mass spectrum: m/z 396 (m+1).

¹³C NMR (125 MHz, CDCl₃) delta 150.4, 140.4, 137.0, 136.5, 134.7, 131.5, 129.5, 128.6, 128.4, 126.2, 122.9, 120.9, 119.9, 119.7, 118.5, 109.5, 100.7, 55.2, 51.0, 46.7, 46.2, 13.1 ppm.

### EXAMPLE 10

1-[2′-(4-Methyl-piperazin-1-yl)-biphenyl-4-ylmethyl]-1H-pyrrolo[2.3-b]pyridine

Step 1

1-(2′-Bromo-biphenyl-4-Vlmethyl)-1H-pyrrolo[2.3-b]pyridine

The title compound was prepared in an analogous fashion to Example 7, Step 2 utilizing sodium hydroxide (667 mg), water (750 ul), 7-azaindole (112 mg, 0.95 mmol), tetrabutylammonium hydrogensulfate (16 mg, 0.05 mmol) and the title compound from Example 7, Step 1 (0.95 mmol) dissolved in 4 ml of toluene. Purification by flash column chromatography (40 micron mesh silica gel; elution with ethyl acetate:hexanes,10:90 in volume, to 100% methanol) afforded the title compound as an oil (112 mg, 33% yield).

¹HNMR (400 MHz, CDCl₃) delta 8.36 (m, 1H), 7.94 (m, 1H), 7.63 (m, 1H), 7.24 (m, 9H), 6.5 (m,1H), 5.57 (s, 2H) ppm.

¹³C NMR (125 MHz, CDCl₃) delta 143.15, 142.3, 140.6, 140.5, 137.3, 133.4, 131.5, 131.4, 130.0, 129.3, 129.0, 128.3, 127.6, 127.2, 122.8, 120.8, 116.2, 100.5, 47.8 ppm.

Step 2

1-[2′-(4-Methyl-piperazin-1-yl)-biphenyl-4-ylmethyl]-1H-pyrrolo[2.3-b]pyridine

The title compound was prepared in an analogous fashion to Example 7, Step 3, utilizing the title compound from Example 10, Step 1 (112 mg, 0.31 mmol) in 2.0 ml toluene, 1-methyl piperazine (207 ul, 1.86 mmol), palladium(II)acetate (142 mg, 0.62 mmol), 2,2′-bisdiphenylphosphanyl-[1,1′]binaphthalenyl (386 mg, 0.62 mmol) and sodium-tert-butoxide (238 mg, 2.48 mmol). The reaction mixture was heated at 120° C. for one and one-half hours and stirred at room temperature for 18 hours. The reaction mixture was worked up analogously to Example 7, Step 3, and purification by flash chromatography (40 micron mesh silica gel; elution with methanol; dichloromethane, 6:94 in volume) afforded the title compound as an oil (1.5 mg, 27% yield).

Mass spectrum: m/z 383 (m+1).

¹HNMR (400 MHz, CDCl₃) delta 8.33 (m, 1H), 7.93 (m, 1H), 7.44 (m, 2H), 7.18 (m, 8H), 6.50 (m, 1H), 5.53 (s, 2H), 3.38 (m, 2H), 3.24 (m, 2H), 3.00 (m, 2H), 2.67 (s, 3H), 2.56 (m,2H) ppm.

### EXAMPLE 11

2-Methyl-1-[2′-(4-methyl-piperazin-1-yl)-biphenyl-4-ylmethyl]-1H-benzoimidazole

Step 1

1-(2′-Bromo-biphenyl-4-ylmethyl)-2-methyl-1H-benzoimidazole

Sodium hydride (53 mg, 1.3 mmol) was added to a solution of 2-methylbenzoimidazole (176 mg, 1.3 mmol) in DMF (0.5 ml). The mixture was stirred for ten minutes at room temperature and then heated at 50° C. for ten minutes. The title compound from Example 7, Step 1 (0.95 mmol) dissolved in 0.5 ml DMF was added and the reaction mixture was heated at 50° C. for 18 hours. The cooled reaction mixture was diluted with 10 ml of water and extracted with three 15 ml portions of dichloromethane. The combined organic extracts were dried (Na₂SO₄) and concentrated to an oil. Purification by flash chromatography (40 micron mesh silica gel; elution with methanol: dichloromethane, 3:97 in volume) afforded the title compound as an oil (234 mg, 65% yield).

Mass spectrum: m/z 377, 379 (m, m+2).

¹³C NMR (125 MHz, CDCl₃) delta 152.1, 142.9, 141.9, 140.9, 140.8, 135.4, 133.4, 131.4, 130.2, 129.2, 127.7, 126.1, 126.0, 122.6, 122.3, 119.4, 109.6, 47.1, 14.3 ppm.

Step 2

2-Methyl-1-[2′-(4-methyl-piperazin-1-yl)-biphenyl-4-ylmethyl]-1H-benzoimidazole

The title compound was prepared in an analogous fashion to Example 7, Step 3, utilizing the title compound from Example 11, Step 1 (234 mg, 0.62 mmol) in 3.0 ml toluene, 1-methyl piperazine (416 ul, 13.7 mmol), palladium(II)acetate (28.5 mg, 0.12 mmol), 2,2′-bisdiphenylphosphanyl-[1,1′]binaphthalenyl (77 mg, 0.12 mmol) and sodium-tert-butoxide (358 mg, 3.7 mmol). The reaction mixture was heated at 100° C. for two hours, stirred at room temperature for 18 hours and worked up analogously to Example 7, Step 3. Purification by flash chromatography (40 micron mesh silica gel; elution with methanol: dichloromethane, 5:95 in volume) afforded the title compound as an oil (135 mg, 55% yield).

Tlc R_{f} (silica gel plates; elution with methanol: dichloromethane, 5:95 in volume, UV detection): 0.53.

¹³C NMR (125 MHz, CDCl₃) delta 150.5, 143.0, 141.2, 135.8, 135.7, 134.4, 131.5, 129.7, 129.6, 128.8, 126.5, 122.9, 122.4, 122.2, 119.4, 118.6, 109.7, 55.3, 51.2, 47.3, 46.3, 14.4 ppm.

### Example 12

1-Methyl-4-(4′-[1,2,4]triazol-1-ylmethyl-biphenyl-2-yl)-piperazine

Step 1

1-(2′-Bromo-biphenyl-4-ylmethyl)-1H-[1,2,4]triazole

1,2,4-Triazole sodium salt (236 mg, 2.7 mmol) was added to a solution of the title compound from Example 7, Step 1 (1.9 mmol) in 1.5 ml of DMF and the resulting mixture was heated at 50° C. for 18 hours. The cooled reaction mixture was diluted with 10% aqueous sodium bicarbonate (15 ml) and extracted with three 15 ml portions of dichloromethane. The combined organic layers were dried (Na₂SO₄) and concentrated to an oil. Purification by flash chromatography (10 micron mesh silica gel; elution with methanol: dichloromethane, 3:97 in volume) afforded the title compound as an oil (415 mg, 70% yield).

Mass spectrum: m/z 314, 316 (m, m+2).

¹³C NMR (125 MHz, CDCl₃) delta 152.5, 143.5, 141.9, 141.6, 134.2, 133.4, 131.4, 130.3, 129.3, 127.8, 127.7, 122.6, 53.4 ppm.

Step 2

1-Methyl-4-(4′-[1,2,4]triazol-1-ylmethyl-biphenyl-2-yl)-piperazine

The title compound was prepared in an analogous fashion to Example 7, Step 3, utilizing the title compound from Example 12, Step 1 (207 mg, 0.66 mmol) in 3.0 ml toluene, 1-methyl piperazine (442 ul, 4.0 mmol), palladium(II)acetate (30 mg, 0.13 mmol), 2,2′-bisdiphenylphosphanyl-[1,1′]binaphthalenyl (81 mg, 0.13 mmol) and sodium-tert-butoxide (384 mg, 4.0 mmol). The reaction mixture was heated at 100° C. for three hours, stirred at room temperature for 18 hours and worked up analogously to Example 7, Step 3. Purification by flash chromatography (40 micron mesh silica gel; elution with methanol: dichloromethane, 10:90 in volume) afforded the title compound as an oil (9 mg, 4.1% yield).

Mass spectrum: m/z 334 (m+1).

¹³C NMR (125 MHz, CDCl₃) delta 152.4, 150.5, 143.3, 141.9, 134.2, 133.0, 131.6, 129.8, 128.9, 128.1, 123.0, 118.7, 55.3, 53.7, 51.2, 46.3 ppm.

### Example 13

3-(4′-[1,2,4]Triazol-1-ylmethyl-biphenyl-2-yl)-piperidine

Step 1

3-(4′-[1,2,4]Triazol-1-ylmethyl-biphenyl-2-yl)-pyridine

To a solution of the title compound from Example 12, Step 1 (207 mg, 0.67 mmol) in 4 ml of THF were added diethyl(3-pyridyl)borane (110 mg, 0.75 mmol), bis(triphenylphosphine)palladium(II) chloride (71 mg, 0.01 mmol), and a solution of sodium carbonate (320 mg, 3 mmol) in 2 ml of water.

The resulting mixture was heated at 80° C. for six hours and then stirred at room temperature for 18 hours. Following dilution with 15 ml of water, the mixture was extracted with three 20 ml portions of dichloromethane. The combined organic layers were dried (Na₂SO₄) and concentrated to an oil. Purification by flash chromatography (40 micron mesh silica gel; elution with methanol: dichloromethane, 5:95 in volume) afforded the title compound as an oil (178 mg, 85% yield).

Mass spectrum: m/z 313 (m+1).

¹H NMR (400 MHz, CDCl₃) delta 8.41 (d, 1H, J=1), 8.39 (d, 1H, J=1), 8.01 (s, 1H), 7.92 (s, 1H) 7.40 (m, 5H), 7.08 (m, 5H) 5.26 (s, 1H) ppm.

Step 2

3-(4′-f 1,2,4]Triazol-1-ylmethyl-biphenyl-2-yl)-piperidine

A 1M solution of lithium triethylborohydride (980 ul, 0.98 mmol) was added to a solution of the title compound from Example 13, Step 1. The mixture was stirred at room temperature for 30 min and then another portion of lithium triethylborohydride (980 ul, 0.98 mmol) was added. After stirring at room temperature for two hours, the reaction was quenched by dropwise addition of 100 ul of methanol. After stirring for one hour at room temperature the reaction mixture was diluted with 10 ml of sodium carbonate and extracted with three 15 ml portions of dichloromethane. The combined organic extracts were dried (Na₂SO₄) and concentrated to an oil. Purification by flash chromatography (40 micron mesh silica gel; elution with ammonium hydroxide: methanol: dichloromethane, 0.5:5:94.5 in volume) afforded the title compound as an oil (29 mg, 33% yield).

Mass spectrum: m/z 319 (m+1).

¹³C NMR (125 MHz, CDCl₃) delta 152.5, 152.4, 143.4, 142.5, 142.4, 141.2, 133.5, 130.3, 130.1, 127.9, 126.8, 126.1, 53.9, 53.5, 46.8, 40.2, 33.0, 27.2.

### Example 14

3-[4′-(2-Ethyl-pyrrol-1-ylmethyl)-biphenyl-2-yl]-piperidine

Step 1

1-(2′-Bromo-biphenyl-4-ylmethyl)-2-ethyl-1H-pyrrole

The title compound was prepared in an analogous fashion to Example 7, Step 2 utilizing sodium hydroxide (1.52 g), water (1.5 ml), 2-ethylpyrrole (181 mg, 1.9 mmol), tetrabutylammonium hydrogensulfate (32 mg, 0.01 mmol), and the title compound from Example 7, Step 1 (1.9 mmol) in 6 ml of toluene. The reaction mixture was heated at 50° C. for 18 hours and worked up in the same fashion as Example 7, Step 2. Purification by flash chromatography (40 micron silica gel; elution with ethyl acetate:hexanes, 3:97) afforded the title compound as an oil (88 mg, 14% yield).

Tlc R_{f} (silica gel plates; elution with ethyl acetate:hexanes, 3:97 in volume; UV detection): 0.58.

¹H NMR (400 MHz, CDCl₃) delta 7.69 (m, 1H), 7.35 (m, 5H), 7.22 (m, 1H), 7.07 (m, 2H), 6.72 (m, 1H), 6.21 (m, 1H), 6.03 (m, 1H), 5.10 (s, 2H), 2.54 (q, 2H, J=7.5), 1.25 (t, 3H, J=7.5) ppm.

Step 2

3-[4′-(2-Ethyl-pyrrol-1-ylmethyl)-biphenyl-2-yl]-pyridine

The title compound was prepared in an analogous fashion to Example 13, Step 1 utilizing the title compound from Example 14, Step 1 (88 mg, 0.26 mmol dissolved in 2.5 ml THF, diethyl-3-pyridylborane (44 mg, 0.30 mmol), bis(triphenylphosphine)palladium(II) chloride (26 mg, 0.04 mmol), and sodium carbonate (124 mg, 1.2 mmol) dissloved in 0.75 ml water. The reaction mixture was heated at 75° C. for four hours and stirred at room temperature for 18 hours. Work up in a similar fashion to Example 13, Step 1 afforded an oil which was purified by flash column chromatography (40 micron mesh silica gel; elution with ethyl acetate:hexanes, 30:70 in volume) to yield the title compound as an oil (60 mg, 69% yield).

Tlc R_{f} (silica gel plates; elution with ethyl acetate:hexanes, 30:70 in volume; UV detection): 0.46.

¹³C NMR (125 MHz, CDCl₃) delta 150.6, 147.9, 140.7, 140.0, 137.3, 137.2, 137.0, 135.3, 130.9, 130.7, 130.5, 128.6, 128.1, 126.4, 122.9, 121.1, 121.0, 107.2, 105.3, 50.1, 19.6, 13.1 ppm.

Step 3

3-[4′-(2-Ethyl-pyrrol-1-ylmethyl)-biphenyl-2-yl]-piperidine

A 1M solution of lithium triethylborohydride in THF (629 ul, 0.63 mmol) was added to a solution of the title compound from Example 14, Step 2 (60 mg, 0.18 mmol) in 1.5 ml of THF. The reaction mixture was stirred at room temperature for two hours, 1M lithium triethylborohydride (310 ul, 0.31 mmol) was added, and the reaction mixture was stirred at room temperature for one additional hour. Methanol (100 ul) was added in a dropwise fashion to quench the reaction. After the quench was complete, the reaction mixture was stirred ten minutes at room temperature, diluted with saturated aqueous sodium carbonate (10 ml), and extracted with three 15 ml portions of dichloromethane. Purification by flash chromatography (40 micron mesh silica gel; elution with ammonium hydroxide: methanol: dichloromethane, 0.5:5:94.5 in volume) afforded the product as an oil (16 mg, 26% yield).

Mass spectrum: m/z 345 (m+1).

¹³C NMR (125 MHz, CDCl₃) delta 142.5, 141.6, 141.0, 137.3, 135.4, 130.5, 129.7, 127.7, 126.7, 126.3, 126.0, 121.1, 107.3, 105.2, 53.9, 50.1, 46.8, 40.2, 33.0, 27.3, 19.7, 13.1 ppm.

### EXAMPLE 15

3-(4′-Pyrazol-1-ylmethyl-biphenyl-2-yl)-piperidine

Step 1

1-(2′-Bromo-biphenyl-4-ylmethyl)-1H-pyrazole

The title compound was prepared in an analogous fashion to Example 7, Step 2, utilizing sodium hydroxide (1.52 g, 3.8 mmol), water (1.5 ml), pyrazole (258 mg, 3.8 mmol), tetrabutylammonium hydrogensulfate (32 mg, 0.1 mmol) and the title compound from Example 7, Step 1 (1.9 mmol). The reaction mixture was heated for 18 hours and worked up in the same fashion as Example 7, step 2, to afford product (547 mg, 93% yield) which was utilized without further purification in Step 2.

¹H NMR (400 MHz, CDCl₃) delta 7.63 (m, 1H), 7.55 (m, 1H), 7.44 (m, 1H), 7.28 (m, 7H), 6.29 (m, 1H), 5.37 (s, 2H) ppm.

Step 2

3-(4′-Pyrazol-1-ylmethyl-biphenyl-2-yl)-pyridine

The title compound was prepared in an analogous manner to Example 13, Step 1, utilizing the title compound from Example 15, Step 1 (547 mg, 1.74 mmol), dissolved in 14 ml THF, diethyl-3-pyridylborane (295 mg, 2.0 mmol), bis(triphenylphosphine)palladium(II) chloride (183 mg, 0.26 mmol) and sodium carbonate (829 mg, 7.8 mmol) dissolved in 4 ml of water. The reaction mixture was heated at 75° C. for 18 hours and worked up in the same fashion as Example 13, Step 1. The crude material was purified by flash chromatography (40 micron mesh silica gel; elution with methanol: dichloromethane, 3:97 in volume) to afford the title compound as an oil (447 mg, 82% yield).

Mass spectrum: m/z 312 (m+1).

¹³C NMR (125 MHz, CDCl₃) delta 150.3, 147.7, 140.6, 140.5, 139.9, 139.8, 137.6, 136.9, 135.6, 132.2, 130.5, 129.5, 128.7, 127.5, 123.1, 106.3, 106.1, 55.7 ppm.

Step 3

3-(4′-Pyrazol-1-ylmethyl-biphenyl-2-yl)-piperidine

A 1M solution of lithium triethylborohydride (5.02 ml, 5.02 mmol) was added to a solution of the title compound from Example 15, Step 2, and the resulting mixture was stirred at room temperature for three hours. Methanol (200 ul) was added dropwise to quench the reaction and the mixture was stirred at room temperature for 30 minutes after the quench was complete. After dilution with 30 ml of aqueous saturated sodium carbonate, the mixture was extracted with three 30 ml portions of dichloromethane. The combined organic layers were dried (Na₂SO₄) and concentrated to an oil. Purification by flash chromatography (40 micron mesh silica gel; elution with ammonium hydroxide:methanol:dichloromethane, 0.5:5:94.5 in volume) afforded the title compound as an oil (80 mg, 18% yield).

Mass spectrum: m/z 318 (m+1).

¹³C NMR (400 MHz, CDCl₃) delta 142.2, 141.6, 141.5, 139.9, 135.6, 130.5, 129.8, 129.7, 127.8, 127.5, 126.7, 126.1, 106.2, 55.8, 53.6, 46.6, 40.0, 32.8, 27.0 ppm.

### EXAMPLE 16

3-(4′-Pyrrol-1-ylmethyl-biphenyl-2-yl)-piperidine

Step 1

3-(4′-Pyrrol-1-ylmethyl-biphenyl-2-yl)-pyridine

The title compound was prepared in an analogous fashion to Example 13, Step 1 utilizing the compound from Example 8, Step 1 (123 mg, 0.39 mmol) dissolved in 3 ml of THF, diethyl-3-pyridylborane (66 mg, 0.45 mmol), bis(triphenylphosphine)palladium(II) chloride (41 mg, 0.06 mmol) and a solution of sodium carbonate (186 mg, 1.76 mmol) in 1 ml of water. The reaction mixture was heated at 75° C. for 18 hours. The reaction was worked up in a similar fashion to Example 13, Step 1, and the crude material was purified by flash chromatography (40 micron mesh silica gel; elution with methanol: dichloromethane, 4:96 in volume) affording the product as an oil (71 mg, 59% yield).

Mass spectrum: m/z 311 (m+1).

¹³C NMR (125 MHz, CDCl₃) delta 150.6, 147.9, 140.7, 140.4, 137.4, 137.1, 137.0, 131.0, 130.8, 130.5, 128.7, 128.2, 127.0, 123.1, 121.5, 108.8, 108.7, 53.2 ppm.

Step 2

3-(4′-Pyrrol-1-ylmethyl-biphenyl-2-yl)-piperidine

A 1M solution of lithium triethylborohydirde (630 ul, 0.63 mmol) was added to a solution of the title compound from Example 16, Step 1 (57 mg, 0.18 mmol) in 1 ml THF. After stirring at room temperature for two hours, methanol (100 ul) was added in a dropwise fashion to quench the reaction. After dilution with 8 ml aqueous saturated sodium carbonate, the mixture was extracted with three 15 ml portions of dichloromethane. The combined organic extracts were dried (Na₂SO₄) and concentrated to an oil. Purification by flash column chromatography (40 micron mesh silica gel; elution with ammonium hydroxide:methanol:dichloromethane, 0.5:5:94.5, in volume) afforded the title compound as an oil (24 mg, 42% yield)

Mass spectrum: m/z 317 (m+1).

¹³C NMR (125 MHz, CDCl₃) delta 141.7, 141.1, 137.2, 130.5, 129.7, 127.9, 126.9, 126.6, 126.3, 121.5, 108.8, 53.3, 53.0, 46.2, 39.4, 32.5, 26.4 ppm.

### EXAMPLE 17

1-(2′-Piperidin-3-yl-biphenyl-4-ylmethyl)-1H-indole

Step 1

1-(2′-Bromo-biphenyl-4-ylmethyl)-1H-indole

The title compound was prepared in an analogous manner to Example 7, Step 2, utilizing sodium hydroxide (541 mg), water (600 ul), tetrabutylammonium hydrogensulfate (13 mg, 0.04 mmol), and a solution of the title compound from Example 7, Step 1 (264 mg, 0.77 mmol) in toluene (2.5 ml). The reaction mixture was heated at 35° C. for 18 hours and worked up in a similar fashion to Example 7, Step 2. Purification by flash chromatography (40 micron mesh silica gel; elution with ethyl acetate:hexanes, 3:97 in volume) afforded the title compound (154 mg, 54% yield).

Mass spectrum: m/z 362,364 (m, m+2).

¹H NMR (400 MHz, CDCl₃) delta 7.68 (m, 1H), 7.54 (m, 2H), 7.42 (m, 1H), 7.32 (m, 2H), 7.22 (m, 7H), 6.59 (m, 1H), 5.40 (m, 2H) ppm.

Step 2

1-(2′-Pyridin-3-yl-biphenyl-4-ylmethyl)-1H-indole

To a solution of the title compound from Example 17, Step 1 (124 mg, 0.34 mmol) in 3 ml THF were added diethyl-3-pyridylborane (57 mg, 0.39 mmol), bis(triphenylphosphine)palladium(II) chloride (36 mg, 0.05 mmol), and a solution of sodium carbonate (166 mg, 1.6 mmol) in 1 ml of water. The reaction mixture was heated at 80° C. for 18 hours, cooled, diluted with 8 ml of water, and extracted with three 15 ml portions of dichloromethane. The combined organic extracts were dried (Na₂SO₄) and concentrated to an oil. Purification by flash chromatography (40 micron mesh silica gel; elution with methanol: dichloromethane, 4:96 in volume) afforded the title compound as an oil (17 mg, 14% yield).

Mass spectrum: m/z 361 (m+1).

¹H NMR (400 MHz, CDCl₃) delta 8.42 (m,2H), 7.63 (m,1H), 7.4 (m,5H), 7.08 (m, 9H), 6.54 (m, 1H), 5.28 (s, 2H) ppm.

Step 3

1-(2′-Piperidin-3-yl-biphenyl-4-ylmethyl)-1H-indole

The title compound from example 17, Step 2 (17 mg, 0.05 mmol) was treated with a 1M solution of lithium triethylborohydride (165 ul, 0.17 mmol) and stirred at room temperature for 20 minutes. The reaction mixture was treated with an additional portion of lithium triethylborohydride (165 uL, 0.17 mmol), stirred 20 minutes at room temperature and quenched by dropwise addition of 100 ul of methanol. The quenched mixture was diluted with 5 ml of aqueous saturated sodium carbonate and extracted with three 8 ml portions of dichlomethane. The combined organic extracts were dried (Na₂SO₄) and concentrated to an oil. Purification by flash column chromatography (40 micron mesh silica gel; elution with ammonium hydroxide, methanol, dichloromethane, 0.5:5:94.5 in volume) afforded the title compound as an oil (1.5 mg, 9% yield).

Mass spectrum: m/z 367 (m+1).

Tic R_{f} (silica gel plates; elution with ammonium hydroxide:methanol:dichloromethane, 0.5:5:94.5; UV detection) 0.31.

¹HNMR (400 MHz, CDCl₃) delta 7.66 (m, 1H), 7.41 (m,1H), 7.18 (m, 11H), 6.56 (m, 1H), 5.38(s, 2H), 3.08 (m, 1H), 2.89 (m, 2H), 2.58 (m, 2H), 1.82(m, 1H), 1.72 (m, 1H), 1.52 (m, 2H) ppm.

### EXAMPLE 18

The following compounds are prepared using the procedures described herein:

1-{4-[2-(4-Methyl-piperazin-1-yl)-pyridin-3-yl]-benzyl}-1H-benzoimidazole;

5-Chloro-1-{4-[2-(4-methyl-piperazin-1-yl)-pyridin-3-yl]-benzyl}-1H-benzoimidazole;

6-Chloro-1-{4-[2-(4-methyl-piperazin-1-yl)-pyridin-3-yl]-benzyl}-1H-benzoimidazole;

1-[3-(4-Imidazol-1-ylmethyl-phenyl)-pyridin-2-yl]-4-methyl-piperazine;

1-{4-[2-(4-Methyl-piperazin-1-yl)-pyridin-3-yl]-benzyl}-1H-indole;

5-Fluoro-1-{4-[2-(4-methyl-piperazin-1-yl)-pyridin-3-yl]-benzyl}-1H-indole;

5-Bromo-1-{4-[2-(4-methyl-piperazin-1-yl)-pyridin-3-yl]-benzyl}-1H-indole;

5-Methyl-1-{4-[2-(4-methyl-piperazin-1-yl)-pyridin-3-yl]-benzyl}-1H-indole;

1-Methyl-4-[3-(4-pyrrol-1-ylmethyl-phenyl)-pyridin-2-yl]-piperazine;

2-Methyl-1-{4-[2-(4-methyl-piperazin-1-yl)-pyridin-3-yl]-benzyl}-1H-indole;

1-{4-[2-(4-Methyl-piperazin-1-yl)-pyridin-3-yl]-benzyl}-1H-pyrrolo[2,3-b]pyridine;

2-Methyl-1-{4-[2-(4-methyl-piperazin-1-yl)-pyridin-3-yl]-benzyl}-1H-benzoimidazole;

and 1-Methyl-4-[3-(4-[1,2,4]triazol-1-ylmethyl-phenyl)-pyridin-2-yl]-piperazine.

What is claimed is:

1. A compound of the formula 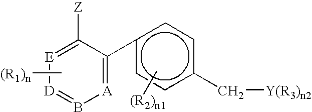
or pharmaceutically acceptable salts thereof wherein 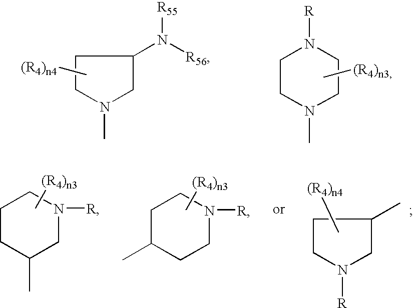
A, B, D, E are independently CH or N, with at most two of A, B, D, and E being N;
each R, R₁, R₂, R₃ and R₄ are independently hydrogen loweralkyl which is unsubstituted or substituted with one to four substituents selected from halo, hydroxy, lower alkoxy, lower alkyl, cycloalkyl, cycloalkoxy, cycloalkyl lower alkyl or cycloalkyl lower alkoxy;
Y is a nitrogen containing heteroaryl having 5 to 14 ring atoms and containing at least one ring nitrogen atom and may optionally contain an additional ring heteroatom selected from the group consisting of nitrogen, oxygen and sulfur; said heteroaryl ring containing 5 to 13 ring carbon atoms and up to a total of 20 carbon atoms;
R₅₅ and R₅₆ are independently methyl or ethyl;
n is 0 to 4;
n₁ is 0-5;
n₂ is 0-5;
n₃ is 04; and
n₄ is 0-3.

2. A compound according to claim 1 wherein Z is

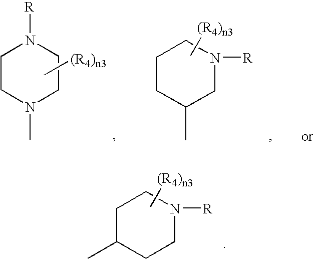

3. A compound according to claim 1 wherein Z is

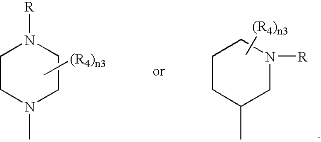

4. A compound according to claim 1 wherein Z is

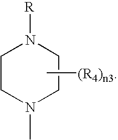

5. A compound according to claim 1 wherein at most one of E, D, B or A is nitrogen.

6. A compound according to claim 1 wherein R, R₁, R₂, R₃ and R₄ are independently hydrogen or lower alkyl which is unsubstituted.

7. A compound according to claim 1 wherein Y is a nitrogen containing heteroaryl containing at most three ring heteroatoms, wherein the ring heteroatoms are nitrogen.

8. A compound according to claim 7 wherein Y contains one or two ring nitrogen atoms.

9. A compound according to claim 1 wherein Y is pyrrolyl, pyrazolyl, triazolyl, imidazolyl, benzoimidazolyl, or indolyl.

10. A compound according to claim 1 having the formula 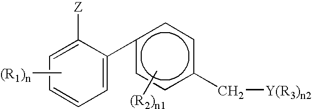
or a pharmaceutically acceptable salt thereof.

11. A compound according to claim 10 wherein Z is

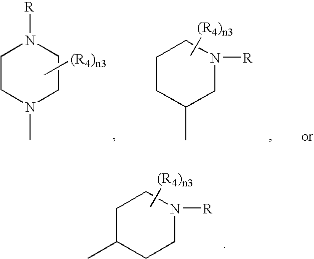

12. A compound according to claim 10 wherein Z is

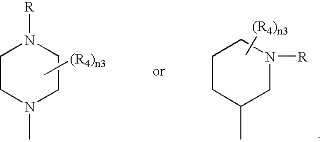

13. A compound according to claim 10 wherein R, R₁, R₂, R₃ and R₄ are independently hydrogen or lower alkyl which is unsubstituted.

14. A compound according to claim 10 wherein Y is a nitrogen containing heteroaryl containing at most three ring heteroatoms, wherein the ring heteroatoms are nitrogen.

15. A compound according to claim 14 wherein Y contains one or two ring nitrogen atoms.

16. A compound according to claim 10 wherein Y is pyrrolyl, pyrazolyl, triazolyl, imidazolyl, benzoimidazolyl or indolyl.

17. A compound according to claim 1 of the formula 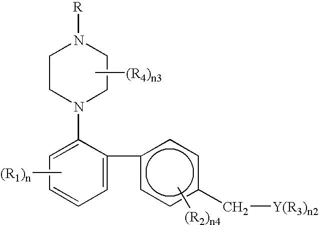
or pharmaceutically acceptable salts thereof.

18. A compound according to claim 17 wherein n, n₁, n₂ and n₃ are 1.

19. A compound according to claim 17 wherein R is lower alkyl and R₁, R₂, R₃ and R₄ are hydrogen or unsubstituted lower alkyl.

20. A compound according to claim 17 wherein Y is pyrrolyl, pyrazolyl, triazolyl, imidazolyl, benzoimidazolyl, or indolyl.

21. A compound according to claim 1 wherein the compound is
1-[2′-(4-Methyl-piperizine-1-yl)-biphenyl-4-ylmethyl]-1H-benzoimidazole;
5-Chloro-1-[2′-(4-methyl-piperazin-1-yl)-biphenyl-4-ylmethyl]-1H-benzoimidazole;
6-Chloro-1-[2′-(4-methyl-piperazin-1-yl)-biphenyl-4-ylmethyl]-1H-benzoimidazole;
1-(4′-Imidazol-1-ylmethyl-biphenyl-2-yl)-4-methyl-piperazine;
1-[2′-(4-Methyl-piperazin-1-yl)-biphenyl-4-ylmethyl]-1H-indole;
5-Fluoro-1-(2′-piperazin-1-yl-biphenyl-4-ylmethyl)-1H-indole;
5-Bromo-1-[2′-(4-methyl-piperazin-1-yl)-biphenyl-4-ylmethyl]-1H-indole;
5-Methyl-1-[2′-(4-methyl-piperazin-1-yl)-biphenyl-4-ylmethyl]-1H-indole;
1-Methyl-4-(4′-pyrrol-1-ylmethyl-biphenyl-2-yl)-piperazine;
2-Methyl-1-[2′-(4-methyl-piperazin-1-yl)-biphenyl-4-ylmethyl]-1H-indole;
1-[2′-(4-Methyl-piperazin-1-yl)-biphenyl-4-ylmethyl]-1H-pyrrolo[2,3-b]pyridine;
2-Methyl-1-[2′-(4-methyl-piperazin-1-yl)-biphenyl-4-ylmethyl]-1H-benzoimidazole;
1-Methyl-4-(4′-[1,2,4]triazol-1-ylmethyl-biphenyl-2-yl)-piperazine;
3-(4′-[1,2,4]Triazol-1-ylmethyl-biphenyl-2-yl)-piperidine;
3-[4′-(2-Ethyl-pyrrol-1-ylmethyl)-biphenyl-2-yl]-piperidine;
3-(4′-Pyrazol-1-ylmethyl-biphenyl-2-yl)-piperidine;
3-(4′-Pyrrol-1-ylmethyl-biphenyl-2-yl)-piperidine;
1-(2′-Piperidin-3-yl-biphenyl-4-ylmethyl)-1H-indole;
1-{4-[2-(4-Methyl-piperazin-1-yl)-pyridin-3-yl]-benzyl}-1H-benzoimidazole;
5-Chloro-1-{4-[2-(4-methyl-piperazin-1-yl)-pyridin-3-yl]-benzyl}-1H-benzoimidazole;
6-Chloro-1-{4-[2-(4-methyl-piperazin-1-yl)-pyridin-3-yl]-benzyl}-1H-benzoimidazole;
1-[3-(4-Imidazol-1-ylmethyl-phenyl)-pyridin-2-yl]-4-methyl-piperazine;
1-{4-[2-(4-Methyl-piperazin-1-yl)-pyridin-3-yl]-benzyl}-1H-indole;
5-Fluoro-1-{4-[2-(4-methyl-piperazin-1-yl)-pyridin-3-yl]-benzyl}-1H-indole;
5-Bromo-1-{4-[2-(4-methyl-piperazin-1-yl)-pyridin-3-yl]-benzyl}-1H-indole;
5-Methyl-1-{4-[2-(4-methyl-piperazin-1-yl)-pyridin-3-yl]-benzyl}-1H-indole;
1-Methyl-4-[3-(4-pyrrol-1-ylmethyl-phenyl)-pyridin-2-yl]-piperazine;
2-Methyl-1-{4-[2-(4-methyl-piperazin-1-yl)-pyridin-3-yl]-benzyl}-1H-indole;
1-{4-[2-(4-Methyl-piperazin-1-yl)-pyridin-3-yl]-benzyl}-1H-pyrrolo[2,3-b]pyridine;
2-Methyl-1-{4-[2-(4-methyl-piperazin-1-yl)-pyridin-3-yl]-benzyl}-1H-benzoimidazole; or
1-Methyl-4-[3-(4-[1,2,4]triazol-1-ylmethyl-phenyl)-pyridin-2-yl]-piperazine or a
pharmaceutically acceptable salt thereof;

22. A pharmaceutical composition comprising a therapeutically effective amount of a compound according to claim 1 or a pharmaceutically acceptable salt thereof and a pharmaceutically acceptable carrier therefor.

23. A method for treating a disorder or condition that can be treated by modulating serotonergic neurotransmission in a mammal, comprising administering to a mammal requiring such treatment a serotonin 7 receptor antagonizing or agonizing effective amount of a compound according to claim 1 or a pharmaceutically acceptable salt thereof.

24. A pharmaceutical composition for treating a condition or disorder that can be treated by modulating serotonergic neurotransmission in a mammal, comprising:
a) a pharmaceutically acceptable carrier;
b) a first compound according to claim 1 or a pharmaceutically acceptable salt thereof; and
c) a second compound selected from the group consisting of a 5HT reuptake inhibitor, a 5HT1B receptor antagonist and a NK1 receptor antagonist and pharmaceutically acceptable salts thereof;
wherein the total amount of the first compound or pharmaceutically acceptable salt thereof, and second compound or the pharmaceutically acceptable salt thereof are such that the composition is effective in treating such disorder or condition.

25. A method for treating a disorder or condition that can be treated by modulating serotonergic neurotransmission in a mammal, comprising administering to a mammal requiring such treatment:
a) a compound according to claim 1, or a pharmaceutically acceptable salt thereof; and
b) a second compound selected from the group consisting of 5HT reuptake inhibitor, a 5HT1 B receptor antagonist and an NK1 receptor antagonist and pharmaceutically acceptable salts thereof;
wherein the amounts of the first compound or pharmaceutically acceptable salt thereof or second compound or pharmaceutically acceptable salt thereof are such that the combination is effective in treating such disorder or condition.

26. A method for treating a disorder or condition selected from depression, anxiety, avoidant personality disorder, premature ejaculation, eating disorders, migraine, premenstrual syndrome, premenstrual dysphoric disorder, seasonal affective disorder, bipolar disorder, jet lag, sleep disorder, nocturnal enuresis, and restless leg syndrome in a mammal, comprising administering to a mammal in need of such treatment an effective amount of a compound according to claim 1, or a pharmaceutically acceptable salt thereof, that is effective in treating such disorder or condition.

27. A method according to claim 26 wherein the sleep disorder is circadian sleep rhythms disorder, sleep deprivation, REM sleep disorder, hypersomnia, parasomnia, sleep-wake cycle disorder, a sleep disorder associated with blindness, a sleep disorder associated with obesity, narcolepsy, or a sleep disorder associated with shift work or irregular work schedules.

28. A method of treating a disorder or condition selected from depression, anxiety, avoidant personality disorder, premature ejaculation, eating disorders, migraine, premenstrual syndrome, premenstrual dysphoric disorder, seasonal affective disorder, bipolar disorder, jet lag, sleep disorder, nocturnal enuresis, and restlessleg syndrome in a mammal, comprising administering to a mammal requiring such treatment: a first compound according to claim 1 or pharmaceutically acceptable salt thereof and a second compound selected from the group consisting of a serotonin reuptake inhibitor, a NK1 receptor antagonist and a 5HT1B receptor antagonist and pharmaceutically acceptable salts thereof; wherein the first compound or its pharmaceutically acceptable salt and second compound or its pharmaceutically acceptable salt are present in amounts that render the combination effective in treating such disorder or condition.

29. A method according to claim 28 wherein the sleep disorder is circadian sleep rhythms disorder, sleep deprivation, REM sleep disorder, hypersomnia, parasomnia, a sleep-wake cycle disorder, a sleep disorder associated with blindness, a sleep disorder associated with obesity, narcolepsy, or a sleep disorder associated with shift work or irregular work schedules.

30. A compound of the formula 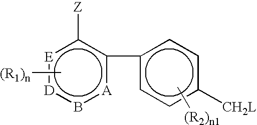
A, B, D, E are independently CH or N, with at most two of A, B, D, and E being N;
each R, R₁, R₂, and R₃ are independently hydrogen loweralkyl which is unsubstituted or substituted with one to four substituents selected from halo, hydroxy, lower alkoxy, lower alkyl, cycloalkyl, cycloalkoxy, cycloalkyl lower alkyl or cycloalkyl lower alkoxy;
L is a leaving group or OH;
n is 0 to 4;
n₁ is 0-5;
n₃ is 0-4; and
n₄ is 0-3.

31. A compound according to claim 30 wherein Z is

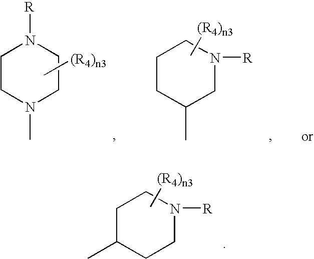

32. A compound according to claim 30 wherein Z is

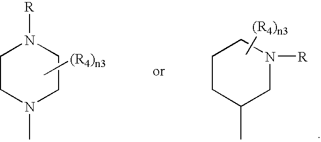

33. A compound according to claim 30 wherein Z is

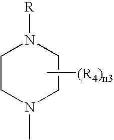

34. A compound according to claim 30 wherein at most one of E, D, B or A is nitrogen.

35. A compound according to claim 30 wherein R, R₁, R₂, R₃ and R₄ are independently hydrogen or lower alkyl which is unsubstituted.

36. A compound of the formula 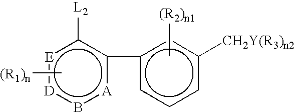
A, B, D, E are independently CH or N, with at most two of A, B, D, and E being N;
each R₁, R₂, and R₃ are independently hydrogen loweralkyl which is unsubstituted or substituted with one to four substituents selected from halo, hydroxy, lower alkoxy, lower alkyl, cycloalkyl, cycloalkoxy, cycloalkyl lower alkyl or cycloalkyl lower alkoxy;
Y is a nitrogen containing heteroaryl having 5 to 14 ring atoms and containing at least one ring nitrogen atom and may optionally contain an additional ring heteroatom selected from the group consisting of nitrogen, oxygen and sulfur; said heteroaryl ring containing 5 to 13 ring carbon atoms and up to a total of 20 carbon atoms;
L₂ is OH;
n is 0 to 4;
n₁ is 0-5; and
n₂ is 0-5.

37. A compound according to claim 36 wherein at most one of E, D, B or A is nitrogen.

38. A compound according to claim 36 wherein Y is a nitrogen containing heteroaryl containing at most three ring heteroatoms, wherein the ring heteroatoms are nitrogen.

39. A compound according to claim 38 wherein Y is a nitrogen containing heteroaryl containing one or two ring nitrogen atoms.

40. A compound according to claim 36 wherein Y is pyrrolyl, pyrazolyl, triazolyl, imidazolyl, benzoimidazolyl or indolyl.
